(19) United States
(12) Patent Application Publication
(10) Pub. No.: US 2023/0099193 A1
(43) Pub. Date: Mar. 30, 2023

(54) PERSONALIZED CANCER LIQUID BIOPSIES USING PRIMERS FROM A PRIMER BANK

(71) Applicant: Pillar Biosciences Inc., Natick, MA (US)

(72) Inventors: Gang Song, Newton, MA (US); Zhaohui Wang, Southborough, MA (US); ShiPing Zou, Medfield, MA (US); Yue Ke, Weston, MA (US)

(21) Appl. No.: 17/936,307

(22) Filed: Sep. 28, 2022

Related U.S. Application Data
(60) Provisional application No. 63/249,925, filed on Sep. 29, 2021.

(51) Int. Cl. C12Q 1/6886; C12Q 1/6869

(52) U.S. Cl. CPC C12Q 1/6886; C12Q 1/6869; C12Q 2600/156; C12Q 2600/16

(57) ABSTRACT

The present invention is directed to a method for detecting somatic mutation in cell free total nucleic acid (cfTNA) in a liquid biological sample by a personalized approach with high sensitivity and specificity. The present invention relates to multiplex amplification of target loci with primers selected from a primer bank for cancer liquid biopsies, including but not limited to minimal residual disease (MRD) monitoring, recurrence monitoring, therapy monitoring, early detecting or screening cancer. Somatic, clonal variants of a patient are first identified by sequencing of the primary tumor and the matched normal sample in the patient. Then customized panel of primer pairs for the patient is selected from a primer bank. Using the selected panel of primer pairs, multiplex polymerase chain reaction and next-generation sequencing are performed on the cfTNA sample from this patient to detect the presence of tumor DNA in the sample.

### REFERENCE TO SEQUENCE LISTING, TABLE OR COMPUTER PROGRAM

This application contains an ST.26 compliant Sequence Listing, which is submitted concurrently in xml format via Patent Center and is hereby incorporated by reference in its entirety. The .xml copy, created on Sep. 26, 2022, is named 113970-8003.US01.xml and is 4,284 KB in size.

### TECHNICAL FIELD

The present invention relates to a method for multiplex amplification of target loci with primers from a primer bank for cancer liquid biopsies, including but not limited to minimal residual disease (MRD) monitoring, recurrence monitoring, therapy monitoring, early detecting or screening cancer by a personalized approach with high sensitivity and specificity. Somatic, clonal variants are first identified by sequencing of the primary tumor and the matched normal sample in a patient. Then a customized panel that includes patient-specific primer pairs for each patient is selected from a primer bank based on the patient's tumor/matched normal sequencing data. Using the selected panel of target-enrichment reagents, multiplex polymerase chain reaction and next-generation sequencing are performed on the plasma cell-free nucleic acid sample from this patient to detect the presence of tumor circulating nucleic acid and its specific mutations in the plasma and monitor the disease.

### BACKGROUND OF THE INVENTION

Data compiled by the American Cancer Society (ACS) show that roughly 40% of the population will develop some sort of invasive malignancy during their lifespan. The 5-year survival rate across all types of cancers is currently between 65 and 70%, but varies greatly depending on cancer type and the stage at which disease is detected and diagnosed. Across all cancer types, the 5 year-survival rate drops from 99% for localized tumors to 27% for metastatic tumors. Early detection, destruction of cancer cells no longer at the primary site and continuous therapy and disease monitoring for each cancer patient are key to improving the cancer survival rate.

For example, general treatment for colorectal cancer (CRC) consists of either radiation or (neoadjuvant) chemotherapy to shrink the tumor, followed by surgery and additional (adjuvant) chemotherapy to kill any occult disease and reduce the risk of recurrence. It is estimated that 17-40% of curatively treated CRC will recur, with high associated mortality.

The concept of MRD is a term commonly used with blood cancers that describes the small fraction of cancer cells that remain or come back after treatment, but it has now been extended to solid tumors as well.

Solid tumors have been shown to compose of regions of different clonality, so a single biopsy may give a biased view of tumor biology. Cell free DNA (cfDNA) fragments have been shown to bear the same unique genetic and epigenetic fingerprint characteristic of the tumor from which they originated. Liquid biopsies, where tumor components are analyzed from patient blood samples, have been shown to give an accurate representation of solid tumor status with a mutation concordance between matched cfDNA and tissue of 83%. A recent survey of NIH clinical trials (www.clinicaltrials.gov) contained over 340 trials that leveraged cfDNA testing, the vast majority to evaluate treatment effectiveness and identify resistance mechanisms

Currently, the most clinically relevant use of sequence information from liquid biopsies is therapy selection, especially for patients with tumors that are difficult to biopsy or whose health being too compromised to undergo surgery. The approach also leads itself to longitudinal monitoring for treatment efficacy and the development of drug resistance. PHARMGKB, a database of annotated database of drug labeling maintained by Shriram Center for Bioengineering and Chemical Engineering contains about 50 small and large molecule oncology therapeutics where genetic testing is required by drug labelling. Such testing informs treatment selection and monitoring for the presence of resistance mutations. The COSMIC database (cancer.sanger.ac.uk/cosmic/drug resistance) maintained by the Sanger Institute currently lists 28 drugs where resistance mutations have been identified.

Besides therapy selection by liquid biopsy, a recent analysis of over 90 clinical studies focusing on the use of circulating tumor DNA (ctDNA) to monitor and predict treatment response in CRC concludes that the ctDNA analysis has great value in both areas and outperforms conventional blood markers such as carcinoembryonic antigen (CEA). Comparison of the sensitivity of ctDNA and CEA for CRC recurrence has consistently demonstrated superiority of ctDNA assays, regardless of biomarkers applied. One study showed that 79% of the patients who had post-operative ctDNA detected subsequently developed disease recurrence compared to 29% with elevated CEA. The same study showed that ctDNA was more likely to be positive than CEA at the time of radiological recurrence (85% vs 41%, p=0.002). Better sensitivity of ctDNA (somatic mutations) compared to CEA has been reported in all studies for recurrence after treatment, with sensitivities reported for ctDNA of 73-100%, with CEA sensitivities ranging from 41-67%. Monitoring treatment success with ctDNA also allows for earlier detection of disease recurrence, with ctDNA detecting recurrence 5-10 months prior to CT compared to 2-3 months median lead-time for CEA.

Analysis of tumor genetics using Next Generation Sequencing (NGS) has been widely utilized to search for mutations associated with prognosis and treatability, but is immensely challenging technically as only about 0.01% of total circulating DNA is tumor-derived. The concentration of cell-free DNA (cfDNA) is influenced by various physiological and pathologic conditions, and the half-life of ctDNA is short, reported to be between 15 minutes and several hours. In addition, recent reports have also indicated that many mutations detected in cfDNA samples actually arise in peripheral blood mononuclear cells via clonal hematopoiesis.

Natera Inc. has launched Signatera, a personalized, tumor-informed assay optimized to detect ctDNA for MRD assessment and recurrence monitoring for patients previously diagnosed with cancer. A patient-specific panel for each patient is designed on the fly based on the whole exon sequencing (WES) data for each patient tumor. Whereas its broad clinical utility and superior assay performance has been proven by a number of prospective clinical studies, the Signatera test is severely limited by the design on the fly approach. The assay turn-around time is several weeks and each patient specific panel is unlikely to be thoroughly validated prior to its use in patient monitoring. More recently, the Guardant Reveal test using a large cancer mutation panel and a methylation panel shows a sensitivity of 91% and can detect recurrence in colorectal cancer months earlier than the standard-of-care method, CEA tests or imaging. However, Gurardant Reveal's assay performance remains controversial, and is cancer-specific

There exists a need for a new and improved method of robust detection of somatic mutation in cell free total nucleic acids (cfTNA) in patient's liquid sample. The method needs to be specific, sensitive, and ease of use.

### BRIEF DESCRIPTION OF THE DRAWING

FIG. 1 illustrates the workflow of sample-to-VOI (variant of interest) identification and testing cfTNA with primers from an off-the shelf (OTS) primer bank.

FIG. 2 illustrates the formation, amplification, and inhibition of primer dimer. A first forward primer (F1) and the second reverse primer (R2) have a complementary region at their 3′ends. The inhibition of primer dimer formation is by the formation of a stem-loop structure. F1∧ is a partial sequence of the 5′-end portion of the F1 primer that is tagged at the 5′ end of the R2 primer.

FIG. 3 illustrates that the amplification of amplicon 3, the overlapping region, is inhibited by the formation of a stem-loop structure. F1, R1, F2, R2 are gene-specific primers, which are complementary to specific regions of genomic DNA. Tags t1 and t2 are two different to universal tag sequences. Tag t3 can have the same or different sequence as t2. Tag oligomers of t1, t2 and t3 do not bind to the target sequences. Each tag is at the 5′end of each gene-specific primer. F2∧ is a partial sequence of the 5′-end portion of the F2 primer. 1, 2, 3 and 4 indicate the amplification products from the combination of four primers. Amplification of Amplicon 3, the short products from F2 and R1, is inhibited by the formation of a stem-loop structure.

FIG. 4 illustrates a flow chart of obtaining customized panel of primers, pre-validating panel of targeted-enrichment reagents, customizing panel of detection reagents, testing reagent, and delivering to customer.

FIG. 5 shows a flow chart of library preparation including multiplex PCR in one container, purifying, indexing, and quantifying.

FIGS. 6-1 to 6-3 illustrate procedures of unique identification (UID) tagging, polymerase chain reaction (PCR) amplification and indexing.

FIG. 7 shows results of detecting somatic mutations in serially diluted SERASEQ™ ctDNA Reference Material. The detection sensitivity was 0.0125%.

FIGS. 8-1 to 8-46 show a 1,688-amplicon panel targeting 7,118 unique variants of a lung cancer panel. All forward and reverse primers shown in FIG. 8 are target gene-specific primers without tags. *T refers to stem-loop inhibition mediated amplification (SLIMAMP®) tag, a tag added to inhibit the unwanted amplification of overlapping regions of target DNAs in PCR reaction. Y means that SLIMAMP® is needed. N means that no SLIMAMP® tag is needed.

FIG. 9 shows SLIMAMP® tags of 3 pairs of overlapping amplicons. SLIMAMP® tag is added at the 5′-end of the reverse primer of each primer pair. SLIMAMP® tag is a partial sequence of the 5′-end sequences of the second forward primer of each pair (bold and underlined).

### DETAILED DESCRIPTION OF THE INVENTION

### Definition

An “amplicon” is a piece of DNA or RNA that is the source (the template) and/or product of amplification or replication events. In this context, “amplification” refers to the production of one or more copies of a genetic fragment or target sequence, specifically the amplicon. As the product of an amplification reaction, amplicon is used interchangeably with common laboratory terms, such as PCR product.

A “genomic locus,” is a physical site or location within a genome. A genomic locus, to as used herein, refers to a region of interest within a gene.

“In silico” refers to being performed on computer or via computer simulation.

A “hot spot” refers to a region that is frequently mutated in a particular cancer.

A “primer dimer” (PD) is a potential by-product in PCR. A PD consists of primer molecules that are hybridized to each other because of complementary bases in the primers.

A “unique identifier” (UID), or a “Unique molecular identifier” (UMI), is a DNA barcode that is added at the beginning of amplification that indexes amplification products derived from the same parent. UID can be used to correct error and remove variants introduced during PCR and allow increased sensitivity for true variants.

“Molecular residual disease” or “minimal residual disease” refers to a small number of cancer cells left in the body of a patient after treatment. These cells have the potential to come back and cause relapse in the patient.

“Variant allele frequency percentage” (VAF %) is the number of reads containing variant as a fraction of all reads for an amplicon.

“Whole exome sequencing” (WES), is a genomic technique for sequencing all of the protein-coding regions of genes in a genome.

The invention provides a method for detecting circulating tumor total nucleic acids (ctTNAs) in a subject by a personalized approach using pre-made target-enrichment reagents including pre-made primer pairs. The method detects one or more mutations in the cell free total nucleic acids (liquid biopsy) of a subject, which may be used to assess that the cancer has recurred or metastasized in the subject, or to evaluate the efficacy of a treatment. This method may be used in liquid biopsy clinical applications, including therapy monitoring, recurrence monitoring, and early detection of cancer. A liquid biopsy, also known as fluid biopsy or fluid phase biopsy, is the sampling and analysis of non-solid biological tissue, primarily blood.

The present invention provides a method for detecting ctTNAs including ctDNA and/or ctRNA in a patient. Patients suitable for testing by the present method have their whole genome sequence data, WES sequence data, large tumor gene panel sequence data, or specific tumor gene sequence data from a tumor issue available. The sequence data are analyzed using analytical software to determine suitable genomic loci for incorporation into the patient customized test. The selection of target regions of interest is based on the analysis from multiple databases and datasets, including but not limited to COSMIC, TCGA, PCAWG, ICGC and any available non-public database. The databases keep record of cancer patients' genomic data. i.e. which cancer shows which variants. Through the database, the frequency of individual variants in each cancer type and in cancer patients can be summarized.

The method uses reagents for targeted amplification of between at least 2 to 45 distinct target genomic loci. The results are associated with the request ID to provide chain-of-custody throughout the manufacturing process. Test reagents include a large panel of primer pairs covering target genomic regions. The primer bank is optimized to have the highest likelihood of multiple positive observations across all common cancers and covering hot spots or frequently mutated loci of one or more cancers.

Once the targeted genomic loci for the patient's customized test panel have been identified, specific primer pairs are selected from the primer bank as an important component of the test reagents. Other test reagents include, but not limited to, index primers, high fidelity PCT master mix. Then the test proceeds with cfTNA of the patient.

The present invention is directed to a method for detecting circulating tumor nucleic acid (ctTNA) from a liquid biological sample of a patient, including detecting minimal residual disease. A liquid biological sample, for example, includes blood, saliva, urine, sweat, cerebrospinal fluid, plural effusion, etc.

The method comprises the steps of: (a) preparing cell free total nucleic acid (cfTNA) from the sample obtained at one or more timepoints from a patient who had cancer at an initial timepoint; (b) selecting at least 2-45 individualized primer pairs from a primer bank, wherein the selected individualized primer pairs correspond to at least 2-45 genomic loci of one or more cancer genes, each primer pair is designed to amplify its corresponding genomic locus; wherein said at least individualized 2-45 genomic loci are selected in silico by (i) analyzing sequence data of whole genome, or whole exome, or a large gene panel, or specific cancer genes, of a tumor tissue of the patient, at the initial timepoint by a computational approach; and (ii) choosing at least 2-45 genomic loci containing somatic mutations specific for the patient, based on clonality, detectability, and frequency of the mutation; wherein the primer bank is designed to comprise multiple primer pairs to amplify different genomic loci of a human, and the primer pairs in the primer banks are designed to be compatible in a single oligonucleotide pool by minimizing primer dimer interaction and reducing amplification of overlapping regions of amplicons by stem-loop inhibition during a PCR reaction; (c) adding the at least 2-45 individualized primer pairs into the cfTNA and perform gene-specific multiplex PCR reaction to amplify the selected genomic loci; (d) purifying, indexing, quantifying, normalizing, and sequencing the amplified DNA, and (e) analyzing the sequencing data and determining circulating tumor nucleic acid (ctTNA)—positive if variants are detected in at least one of the genomic loci in cfTNA.

In step (a), a liquid biological sample from a patient is collected at one time point or at longitudinal timepoints, for preparing cfDNA, cfRNA or both. Prior to cfTNA isolation, the plasma is separated from whole blood by centrifugation, which separates the plasma from the buffy coat (white blood cells) and red blood cells. The plasma layer is removed from the buffy coat to avoid contamination of cellular DNA into the plasma sample. The recovered plasma fraction is optionally subjected to a second centrifugation at high speed to remove much of the remaining cell debris and protein. The plasma fraction is then extracted to simultaneously recover RNA and DNA (total nucleic acids) using validated laboratory methods. The total nucleic acid extraction may use any of the various commercial kits designed for this purpose (e.g., QIAamp® Circulating Nucleic Acid Kit or Norgen).

In step (b), at least 2-45 individualized primer pairs corresponding to at least 2-45 genomic loci are selected and obtained from a primer bank for the patient. In one embodiment, the least 2-45 individualized genomic loci are 2-45, 2-50, or 2-60, or 2-100, or 2-200, or 2-300 genomic loci, and the least 2-45 individualized primer pairs are the corresponding 2-45, 2-50, or 2-60, or 2-100, or 2-200, or 2-300 primer pairs. Preferably, the at least 2-45 individualized genomic loci are 2-45 or 2-60 genomic loci, and the at least 2-45 individualized primer pairs are the corresponding 2-45 or 2-60 or primer pairs.

Prior to obtaining these individualized primer pairs, DNA sequence data of a tumor tissue of the patient at an initial time point are analyzed and the at least 2-45 genomic loci containing somatic mutations are selected by a computational approach for the subsequent testing and analysis. As described by McGranahan et al (Sci Transl Med 7:283ra54, 2015), clonal and subclonal mutations can be identified within single tumor samples.

The DNA sequence data of a patient may be obtained from whole genome, or whole exome (about 20,000-25,000 genes), or a large gene panel (about 400-500 cancer genes), or specific cancer genomes (e.g., about 1-50, 5-50, or 5-10 cancer genes) of a tumor tissue of the patient.

The DNA sequence data from the patient tumor tissue is optionally compared with the sequence data of a matched germline DNA, at the initial time point by a computational approach. The matched germline DNA may be obtained from a normal tissue, a normal whole blood sample, or white blood cell from the same patient.

Based on quality and coverage depth of the sequencing results of the patients' tumor tissue, a list of somatic single-nucleotide variants (SNVs) or insertion-deletion variants (Indel) specific to the patient are selected.

The initial somatic variants are further prioritized based on a list of criteria including, but not limited to, sequencing quality (quality of the sequencer reads), mapping quality (how confident is the alignment of the mutation), variant frequency (the number of the mutations vs the number of the wild types observed), calling confidence level (overall statistical evaluation based on matched normal sample to evaluate how confident the somatic calling is), variant coverage depth (number of reads that actually contain the variant calls), and read bias (location of the mutation in the reads: the more it is towards the center, the better). The loci with optimum GC content (as close to 50% as possible) and higher specificity (amplicon that does not map to multiple locations) are prioritized as well together with the mutation call prioritization.

In one embodiment, software program that delivers sensitive, robust variant calls, for example, PIVAT® or VERSATILE® (Pillar Biosciences Inc.), is used to compare the DNA sequence data of the tumor tissue and white blood cells of the patient to select a list of somatic variants which are prominent in tumor tissue but not in white blood cells. Such selection is specific to the patient. Rules are then applied to this list to give weight to each variant. Then at least top 2-45, 2-300, 2-200, 2-100, 2-60, 2-50, 2-49, 2-48, 2-47, 2-26, 2-45, 3-10, 3-45, 3-48, 3-50, 5-40, 10-30, 10-35, 10-40, or 15-45 genomic loci are picked as having hot spot mutations specific to the patient's tumor profile. In general, each cancer related gene has 1 to several hundreds of hot spots.

After the at least 2-45 genomic loci are selected, at least 2-45 individualized primer pairs that are capable to amply the at least 2-45 genomic loci of are then selected from primer pairs of a primer bank.

We use the public (TCGA, PCAWG, ICGC, COSMIC) and non-public (CLIA Lab) database to search for biomarkers of common cancers. Many genome loci of common cancers are overlapping. We then design one or more primer banks each comprising multiple primer pairs to amplify different genomic loci of a human, in particular, to amplify one or more hot spots of 400-500 cancer genes. The primer pairs in the primer banks are designed to be compatible in a single oligonucleotide pool by minimizing primer dimer interaction and reducing amplification of overlapping regions of amplicons by stem-loop inhibition during a PCR reaction.

In one embodiment, the primer bank is a physical off-the-shelf (OTS) primer bank that contains primers in a form of pre-made oligonucleotides. These primer pairs are pre-made and pre-validated for amplification of different genomic loci of a human, and they are stand-by and ready for clinical use. Primer pairs from a physical bank can be quickly selected and deployed as individual panels of primer pairs for use, without additional testing of the oligonucleotides. The OTS primer bank may contain 5-5000, 10-5,000, 50-10,000, 500-5,000, 10-10,000 or 50-50,000 primer pairs. For example, the primer pairs in a primer bank are designed to cover at least 80% of a patient of one tumor type with 8 or more mutations, which is sufficient for monitoring MRD.

In another embodiment, the primer bank is a virtue bank in which all the sequences of the primers are designed and exist in silico, for amplification of different genomic loci of a human. In a virtue primer bank, the designed primer pairs can be validated as tests are conducted. Each primer pair only needs to be tested for quality control (QC) one time to verify the effectiveness prior to clinical testing, and then it can be reused without additional QC testing in future occurrences. Virtual bank may not have a fast turnaround time in the beginning; but it can be gradually built to include a larger primer pair pool and it eventually achieves a fast turnaround time. Virtual bank has unlimited number of primer pairs, if desired, to cover entire genome of all living species where PCR amplification is possible.

In a further embodiment, the primer bank is a combined physical bank and virtue bank. A subset of the primer pairs is pre-made and pre-validated in a physical form. The rest of primer pairs are added using the strategy described in virtual bank. The physical bank can be gradually growing by adding new primers from the virtual bank. The combined physical bank and virtue bank provides an initial fast turnaround time with common mutations from the physical bank, and the coverage of additional personal mutation of interest is added from the virtue bank later for a better sensitivity of detection.

After the selection of at least 2-45 genomic loci, at least 2-45 individualized primer pairs that are capable to amply the at least 2-45 genomic loci are then selected from a primer bank by overlapping the primer pairs in the primer bank with the selected genomic loci. If there are any primers that will form dimers, the less important dimers are deleted from the initial selection. Due to the low oligo dimer occurrences in an original primer bank, most of the primers can be selected without the need to delete some primers to avoid primer dimer formation. The selection of primer pairs from a primer bank can use software such as VERSATILE® to identify the best primers that cover the selected variants, without dimer formation.

This selected panel of primer pairs from a primer bank covers hot spots about at least 10-45 target regions of the patient; the target regions are optimized to have the highest likelihood of multiple positive observations across all common cancers or the cancer(s) of interest.

In view of DNA fragmentation observed in clinical specimens, the length of the amplification product is preferred to be short to maximize amplification yield.

Each primer pair has a forward primer and a reverse primer, having a length of ≤125 nucleotides or ≤100 nucleotides in general. Each forward primer or reverse primer contains a gene-specific sequence, which is a target-specific sequence complementary to the target DNA of the selected genomic loci. The gene-specific sequence typically has 6-40, 10-50, 10-40, 10-100, 20-40, or 20-50 nucleotides in length. Each forward primer and each reverse primer typically also contain a tag at the 5′-end of each gene-specific sequence; the tag does not bind to the target DNA sequence, and it contains a priming site for a subsequent amplification. The tag sequences are at least 2 or 3 nucleotides in length, and can be 5-100, 3-40, 10-30, 10-40, 10-50 nucleotides long. In one embodiment, the tag sequences of most or all of the forward primers in a primer bank are the same; and the tag sequences of most or all of the reverse primers in the primer bank are same, and they are referred to as universal tags. Depending on the specific design (e.g. to modify the melting temperature of the amplified DNAs), variable sequences and various lengths may be optionally added to the 5′-end of the universal tags.

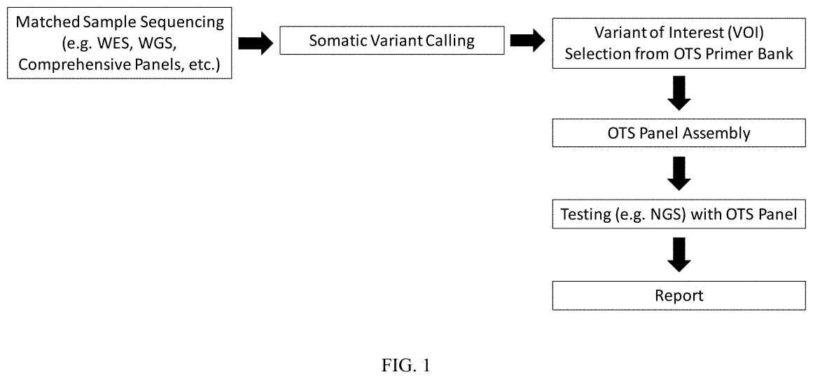

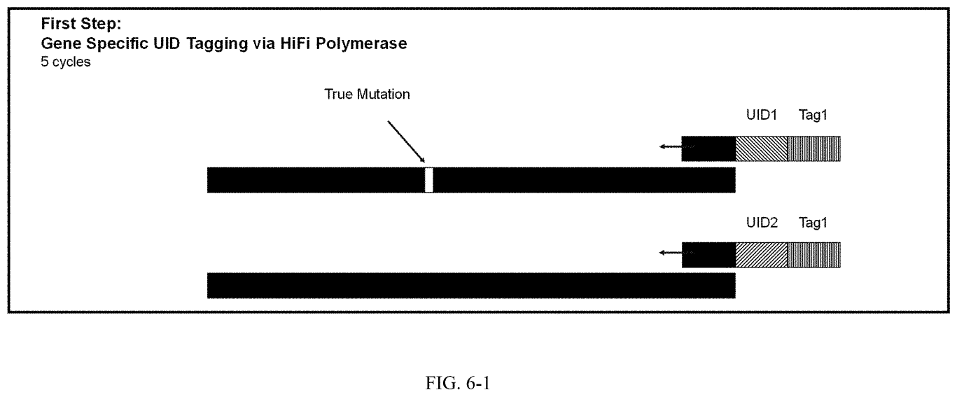

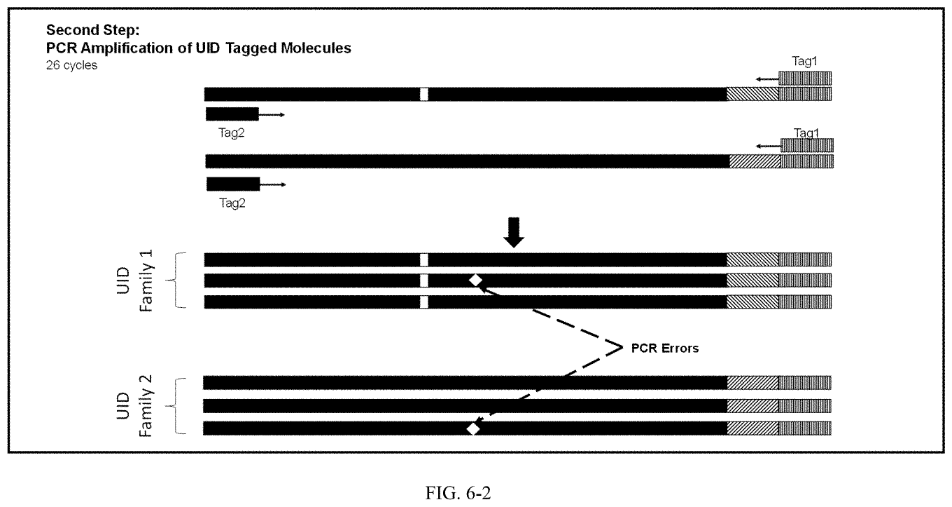

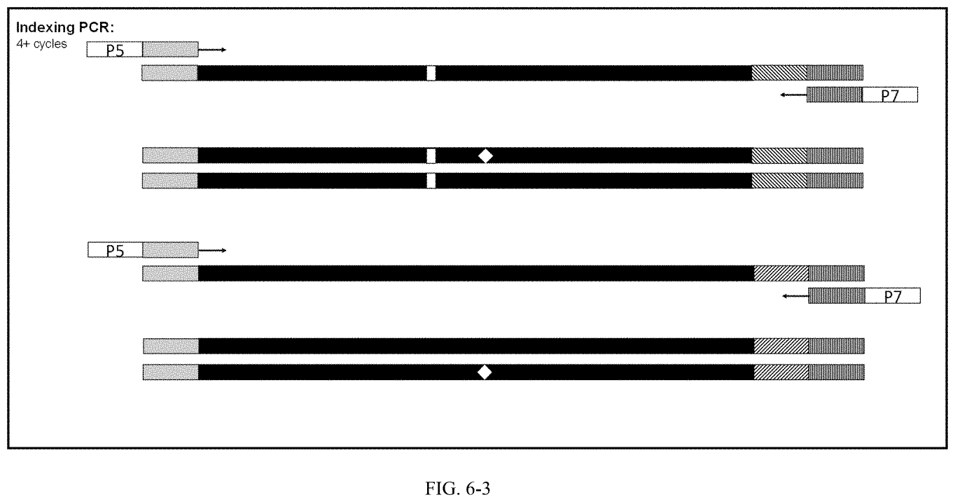

In one embodiment, a UID is added in either a forward primer or a reverse primer to identify individual nucleic acid molecule in the starting sample. In one example, as shown in FIG. 6-1, a reverse primer may comprise from 5′ to 3′ a first segment containing a first tag, which is a priming site for subsequent amplification, a second segment containing a UID to identify each nucleic acid molecule, and a third segment complementary to the target DNA. The UID in general contains 6-14 nucleotides or 8-12 nucleotides. A forward primer may comprise from 5′ to 3′ a first segment containing a second universal tag, which is a priming site for subsequent amplification, and a second segment complementary to the target DNA. In another example, a forward primer may comprise from 5′ to 3′ a first segment containing a first tag, which is a priming site for subsequent amplification, a second segment containing a UID, and a third segment complementary to the target DNA; and a reverse primer may comprise from 5′ to 3′ a first segment containing a second universal tag, and a second segment complementary to the target DNA.

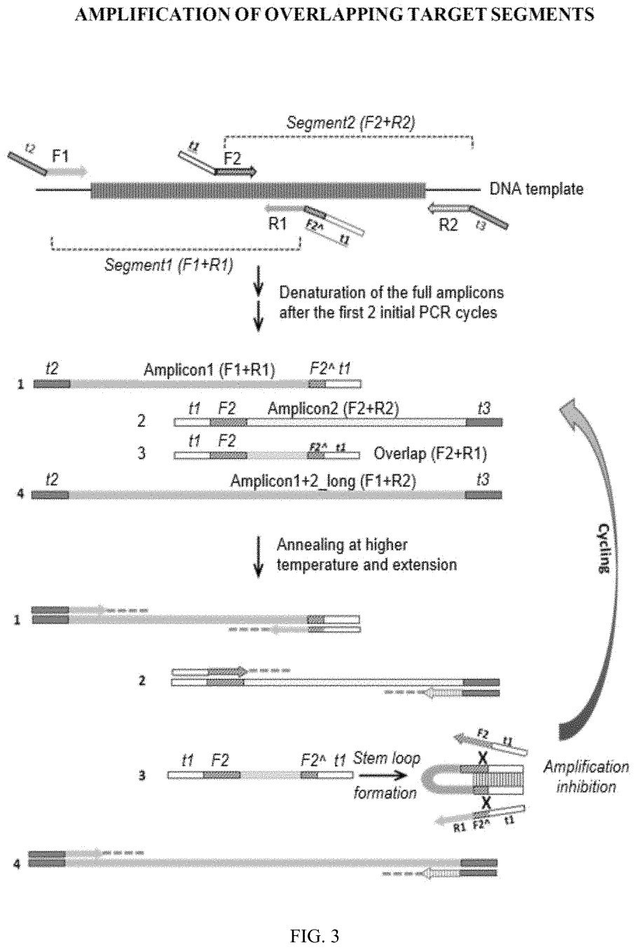

In another embodiment, when overlapping amplicons are needed to cover a long region of interest, the primers are designed according to SLIMAMP® tag strategy (See FIG. 3).

The primer pairs are designed to be compatible in a single oligonucleotide pool by avoiding or inhibiting primer dimer formation and by inhibiting amplification of overlapping regions of amplicons by stem-loop inhibition during a PCR reaction.

In general, the primers in a primer bank are designed and selected to avoid or reduce primer dimer problem. Primer-dimers (primers that are hybridized to each other because of complementary bases in the primers) are identified in an automated manner with a software such as VERSATILE®, and removed from the final pool.

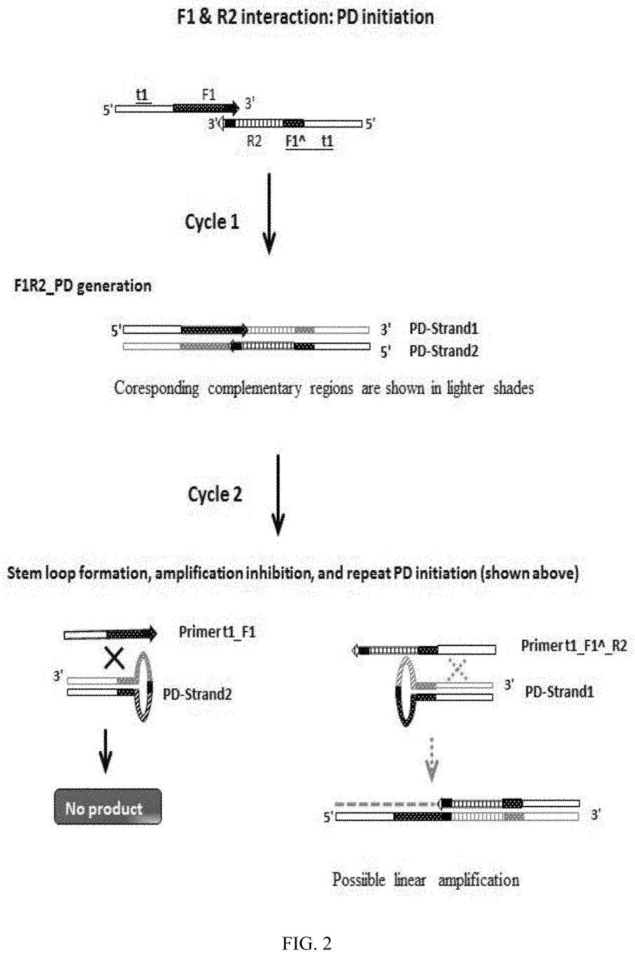

Primer dimer problem can also be resolved according to U.S. Pat. No. 9,605,305, which is incorporated herein by reference in its entirety. The principle of the design to avoid or reduce primer dimer formation during PCR is shown in FIG. 2, which illustrates how to prevent the exponential amplification of a primer dimer. In FIG. 2, a forward primer F1 and a reverse primer R2 have a complementary region at their 3′-ends. After Cycle 1, PD-Strand 1 and PD-Strand 2 are formed. In Cycle 2, on the left side, PD strand 2 forms a stem loop, in which t1 and F1∧ anneal to their complementary counterparts respectively to form a stem, and the remaining nucleotides form a loop. Due to high local concentrations of t1 and F1∧ and their respective complementary counterparts, i.e., they are on the same PD Strand 2 and are close to each other, the formation of the stem loop is more favorable than the annealing with a separate t1F1 primer; therefore, further primer annealing is blocked, and no further amplification product of PD-Strand 2 can be obtained. The presence of F1∧ is important in order to completely block the primer (t1_F1) annealing to PD Strand 2 and then the amplification of PD Strand 2. Without F1∧, the primer t1_F1 may outcompete the stem structure containing only t1 and then anneal to PD Strand 2. With the addition of F1∧, primer t1_F1 can no longer outcompete the stem structure containing t1_F1^{A} for annealing to PD Strand 2.

To achieve the coverage of a continuous sequence over a long target region, amplicons may be overlapped. To avoid amplification of overlapping region of amplicons, the primers are designed according to U.S. Pat. No. 10,011,869, which is incorporated herein by reference in its entirety. The principle of the primer design to inhibit amplification of the overlapping region of two amplicons is shown in FIG. 3. As shown at the lower part of to FIG. 3, amplicon 1 (F1+R1), Amplicon 2 (F2+R2), and Amplicon 4_long (F1+R2) are amplified exponentially by PCR, while the amplification of Amplicon 3_overlap (F2+R1) is inhibited. This is because F2 and R1 gene-specific segments are tagged with the same tag t1, and therefore in the presence of F2∧(a partial sequence of the 5′-end portion of the F2 primer) in between t1 and R1, a strong stem loop structure containing the sequences of t1 and F2∧ forms and prevents the hybridization of primer t1F2 to the amplicon 3 template, which inhibits the further exponential amplification of amplicon 3.

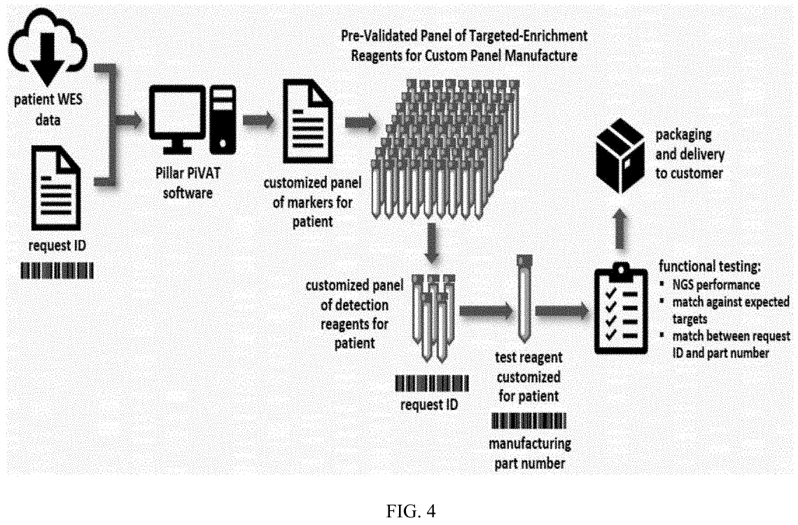

A flow chart of one embodiment for preparing pre-made and pre-validated target enrichment reagents to carry out step (b) is illustrated in FIG. 4.

In one embodiment, all of the at least 2-45 individualized primer pairs are selected and obtained from a primer bank.

In another embodiment, instead of selecting all of the at least 2-45 individualized primer pairs from a primer bank, a small portion of primer pairs, e.g., 1-5 or 1-10 primer pairs can be made specifically for the patient after selecting the 2-45 genomic loci. These specifically made primer pairs can be mixed with the primer pairs from a primer bank to provide one component of the target enrichment reagent. These specifically made primer pairs can be used when some patient's variants are not covered by the primer bank.

In step (c), the at least 2-45 individualized primer pairs are added into cfTNA in one pool and gene-specific multiplex PCR reaction is performed to amplify the selected genomic loci in one single container. The amplification reaction does not require having multiple amplification reactions in separate containers and then pooling the amplified products; this is due to the design of the primers that avoids primer dimer formation and reduces amplification of overlapping regions of amplicons.

In step (d), the amplified DNA are purified, indexed, quantified, and normalized, before being sequenced by a sequencer.

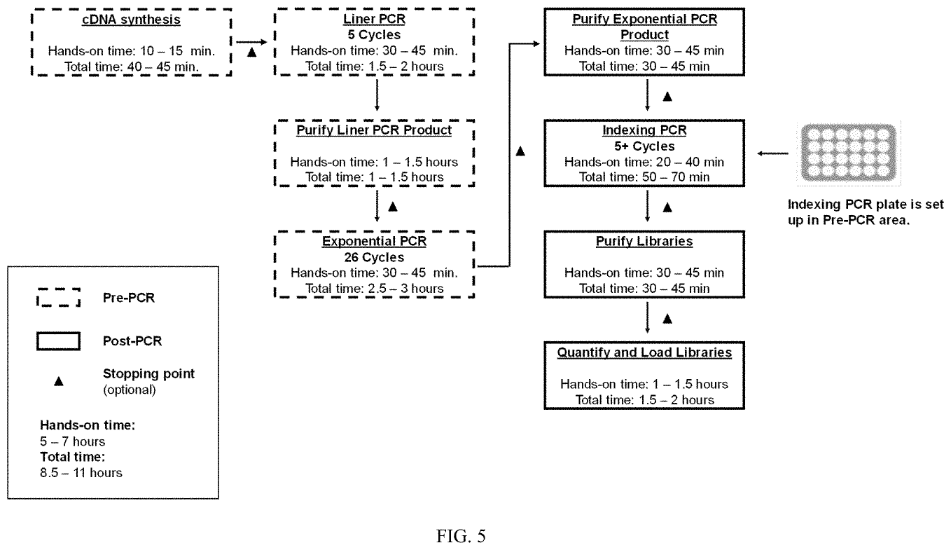

Steps (c) and (d) are illustrated in flow charts of FIGS. 5 and 6.

Library preparation procedure includes three or four steps: (1) conversion of RNA to cDNA (this step is optional), (2) gene-specific multiplex PCR amplification with or without UID (3) a brief indexing PCR amplification that applies the sample-specific barcodes that allow sample pooling, (4) and library normalization and pooling for sequencing. (See FIG. 5)

1. Conversion of cfRNA to complementary DNA (optional step, skip it if use cfDNA as input directly): cDNA is produced from cfRNA using reverse transcriptase and priming with random hexamers. The entire undiluted cDNA reaction can be added to the linear PCR without inhibiting the reaction. Alternatively, with a higher cfRNA input, the cDNA reaction can be diluted with low TE or nuclease-free water. The recommend minimum input is 10 ng of total circulating nucleic acid.

2. Gene-specific multiplex PCR amplification: SLIMAMP® Multiplex PCR is performed with or without UID tags. A randomer tag is added to sample DNA and cDNA molecules by a brief linear PCR. The purpose of the UID tag is to identify members of an amplification cluster that arose from the clonal outgrowth of a single sample nucleic acid molecule. This information is subsequently used to error correct mutations introduced during PCR amplification to allow higher sensitivity sample mutation detection.

3. Universal indexing PCR amplification: SLIMAMP® products are purified with a PCR cleanup procedure. The purified products are then indexed by PCR using indexing primers to enable multiplex sequencing on a sequencer such as NextSeqDx.

4. Library normalization: The indexed libraries are subsequently purified, quantified and normalized for library pooling. The pooled libraries are then run on a sequencer such as NextSeqDx using a paired-end sequencing protocol.

Because it is critical to reduce PCR errors in low-frequency variant detection, to minimize the PCR error, all PCR processes utilize a high-fidelity polymerase with an error rate>100-fold lower than that of Taq DNA Polymerase. Gene-specific primers can be tagged with a Unique Identifier (UID) containing 8-20 random bases, errors from PCR amplification and from the sequencing process can be reduced by calling the consensus bases across all reads within a UID family (See FIGS. 6-1 and 6-2).

The products from PCR are subsequently purified via size selection. After purification, another round of PCR adds index adaptors of P5 and P7 sequences to each library for sample tracking and sequencing on Illumina's flow cells. Those products are further purified and sequenced (FIG. 6-3).

In step (e), the sequence data are analyzed.

The base calls are generated on the sequencing instrument (e.g. MiSeq, NextSeq and NovaSeq) during the sequencing run by Real Time Analysis (RTA) software during primary analysis. After the sequencing run, a software, BCL2FASTQ, is used to perform the initial two steps as described below.

Demultiplexing: Each sample is tagged with unique indexes during the indexing PCR step. The demultiplexing step divides the sequence reads into separate files according to the index information specified in the sample sheet.

FASTQ File Generation: After demultiplexing, on-instrument MiSeq-Reporter generates the FASTQ files that contain the cluster-passing-filter reads for each sample with quality scores and paired-end information.

Subsequently, the FASTQ files are analyzed with Pillar's PIVAT® software that performs the rest of the secondary analysis and reports out detected target variants.

The biomedical information of the patient obtained in step (e) may be used for predicting, prognosing, or diagnosing a disease state of the patient. For example, the biomedical information may be used to determine the efficacy of a drug therapy of the patient, to predict an optimal drug dosage, to recommend one or more therapies, or to recommend a course of treatment of a disease. For example, the method can be used to detect minimal residual disease. After analyzing the sequencing data and determining whether ctTNA is positive or negative in step (e), the present method may further comprise a step (f) for determining a therapy choice or a change in therapy for the patient.

The following examples further illustrate the present invention. These examples are intended merely to be illustrative of the present invention and are not to be construed as being limiting.

### EXAMPLES

### Example 1. Library Preparation Protocol

### Example 1A. Gene-specific PCR (GS-PCR)

1. Prepare a PCR master mix: Vortex and spin the HiFi PCR MMX and oligo pool before use. For each PCR reaction, the volume of each component is listed below in Table 1.

TABLE 1

| Reagent | Volume (μL) |
|---|---|
| HiFi PCR Master Mix (4x) | 12.5 |
| GS-PCR oligo pool | 2.5 |
| Sub-Total | 15.0 |

2. Transfer reagents to PCR plate
a. Transfer 15 μL of master mix to each sample well in a PCR plate. Then, add 35 μL of cfDNA (total 30 ng) to the corresponding wells (Table 2).

TABLE 2

| Reagent | Volume (μL) |
|---|---|
| PCR master mix | 15.0 |
| cfDNA | 35.0 |
| Total | 50.0 |

3. Seal and mix: Carefully seal the reactions and vortex for 10-15 seconds.

4. Spin: Briefly spin the reactions to remove any air bubbles from the bottom of the wells and spin down droplets from the seal or side walls.

5. Perform PCR: Perform the following program with the heated lid on (Table 3).

TABLE 3

| | | |
|---|---|---|
| Temp | Time | # of cycles |

| | | | |
|---|---|---|---|
| 95° C. | 15 | sec | 1 |
| 95° C. | 1 | min | 5 |
| 58° C. | 2 | mins | |
| 60° C. | 4 | mins | |
| 64° C. | 1 | min | |
| 72° C. | 1 | min | |
| 95° C. | 30 | sec | 21 |
| 66° C. | 3 | min | |

| | | |
|---|---|---|
| 8° C. | Hold | Hold |

### Example 1B. Purify the GS-PCR Product

Pre-Purification

Warm AMPure beads: Take out Agencourt AMPure XP beads from 4° C. and incubate at room temperature for at least 30 minutes before use.

GS-PCR Product Purification
1. Briefly spin the samples to remove droplets from the side walls. Carefully remove the seal.
2. Mix beads: Vortex AMPure XP beads thoroughly until all beads are well dispersed.
3. Add beads: Add 75 μL beads to each well. Pipette the mixture up and down 10 times. If bubbles form on the bottom of the wells, briefly spin the samples and mix again.
4. Bind PCR product to beads: Incubate the samples for 5 minutes at room temperature.
5. Separate beads containing PCR product: Place the samples on a magnetic rack until the solution appears clear, which can take up to 5 minutes.
6. Remove supernatant: Carefully remove the supernatant from each well without disturbing the beads from the wall of each well.
7. Wash beads: Leave the samples on the magnetic rack. Add 150 μL of freshly prepared 70% ethanol to each well without disturbing the beads. Incubate 30 seconds, and then remove the supernatant from each well.
8. Second wash: Repeat step 7 for a second 70% ethanol wash. Remove the supernatant from each well. The unused solution of ethanol can be used to purify the libraries after indexing PCR.
9. Remove remaining ethanol wash: Remove trace amounts of ethanol completely from each well. Spin the samples in a benchtop centrifuge for 10-15 seconds, place the samples back on the magnetic rack, and use a 10 or 20 μL tip to remove the remaining ethanol solution at the bottom of the wells.
10. Dry beads: Let the beads air dry at room temperature for 2-5 minutes.
11. Resuspend beads: Remove the samples from the magnetic rack, and immediately resuspend the dried beads in each well using 32 μL nuclease-free water. Gently pipette the suspension up and down 10 times. If bubbles form on the bottom of the wells, briefly spin and mix again.
12. Elute: Incubate the elution at room temperature for 5 minutes to fully elute the product.
13. Separate supernatant containing PCR product: Place the samples on a magnetic rack until the solution appears clear, which can take up to 5 minutes.

### Example 2. Protocols for Indexing PCR

1. Prepare a Master Mix: Vortex and spin the High-Fidelity PCR Master Mix before use. To prepare the PCR master mix, combine the High-Fidelity PCR Master Mix and water sufficient for the samples being processed with overage. Transfer 32 μL of master mix to each sample well in a PCR plate (Table 4).

TABLE 4

| Reagent | Volume (μL) |
|---|---|
| HiFi PCR Master Mix (4x) | 12.5 |
| Nuclease-free water | 19.5 |
| Total | 32.0 |

2. Add indexing primers and purified GS-PCR product to each well:. Add 4 uL of each of the two indexing primers and 10 μL of the Ampure purified GS-PCR supernatant into the wells containing the PCR Master Mix, being sure that no beads are transferred (Table 5).

TABLE 5

| Reagent | Volume (μL) |
|---|---|
| Indices and PCR Master Mix | 32.0 |
| Pi700 Pillar Index | 4.0 |
| Pi500 Pillar Index | 4.0 |
| Purified Gene-specific PCR product | 10.0 |
| Total | 50.0 |

3. Mix and spin: Seal the plates and pulse vortex the sealed reactions on a medium setting for 5-10 seconds to mix. Briefly spin down the reactions to remove any bubbles within the reaction solutions.
4. Perform PCR: Perform the following program with the heated lid on (Table 6).

TABLE 6

| | | |
|---|---|---|
| Temperature | Time | Number of Cycles |

| | | | |
|---|---|---|---|
| 95° C. | 1 | min | 1 |
| 95° C. | 30 | sec | 5* |
| 66° C. | 30 | sec | |
| 72° C. | 60 | sec | |
| 72° C. | 5 | min | 1 |

| | | |
|---|---|---|
| 8° C. | Hold | 1 |

*This number can be varied.

### Example 3. Protocols for Purifying the Libraries

1. Briefly spin the samples to remove any droplets from the side walls. Carefully remove the seal.
2. Mix beads: Vortex the room temperature pre-warmed AMPure XP beads thoroughly until all beads are well dispersed.
3. Add beads: Add 60 μL beads (1.2×beads) to each well. Pipette the mixture up and down 10 times. If bubbles form on the bottom of the wells, briefly spin and mix again.
4. Bind libraries to beads: Incubate the samples for 5 minutes at room temperature to bind the libraries to the beads.
5. Separate libraries on beads: Place the samples on a magnetic rack until the solution appears clear, which can take up to 5 minutes.
6. Remove supernatant: Carefully remove the supernatant from each well without disturbing the beads from the wall of each well.
7. Wash beads: Leave the samples on the magnetic rack. Add 150 μL of freshly prepared 70% ethanol to each well without disturbing the beads. Incubate 30 seconds, and then remove the supernatant from each well.
8. Second wash: Repeat step 7 for a second 70% ethanol wash. Remove the supernatant from each well.
9. Dry beads: Let the beads air dry at room temperature for 2-5 minutes.
10. Resuspend beads: Remove the samples from the magnetic rack and resuspend the dried beads in each well using 32 μL nuclease-free water. Gently pipette the beads suspension up and down 10 times. If bubbles form on the bottom of the wells, briefly spin and mix again.
11. Elute libraries: Incubate the resuspended beads at room temperature for 5 minutes to elute the final libraries.
12. Separate libraries from beads: Place the elution on the magnetic rack at room temperature until the solution appears clear. Transfer 30 μL of clear supernatant from each well of the PCR plate or tubes to the corresponding well of a new plate or tube.
13. Quantitation: Analyze an aliquot of each library according to Example 4.

### Example 4. Protocols for Qubit Quantitation of Purified Libraries

1. Prepare buffer with dye: Dilute the Qubit dsDNA HS reagent 1:200 in Qubit dsDNA HS buffer. Vortex briefly to mix Qubit working solution. For example, 2000 μL is sufficient buffer for 10 readings (8 samples+2 standards). Combine 1990 μL of Qubit dsDNA HS buffer and 10 μL HS reagent. Add reagent overage appropriately
2. Label tubes: Set up 0.5 mL Qubit tubes for standards and samples. Label the tube lids.
3. Prepare standards: Transfer 190 μL of Qubit working solution into two tubes for standard 1 and standard 2, and then add 10 μL of each standard to the corresponding tube.
4. Prepare samples: Transfer 198 μL of Qubit working solution to each tube, and then add 2 μL of each sample to the tube (1:100 dilution).
5. Mix and spin: Mix the tubes by vortexing and then spinning the tubes briefly.
6. Incubate the tubes at room temperature for 2 minutes.
7. Measure concentration: Measure the concentration of each sample on the Qubit 2.0 Fluorometer per the Qubit User Guide. Use the dsDNA High Sensitivity assay to read standards 1 and 2 followed by the samples.
8. If any sample concentrations are above the linear range of the instrument, prepare a new dilution using 199 μL Qubit buffer with dye and 1 μL sample (1:200 dilution). Repeat steps 5-7.
9. Calculate concentration: 1 ng/μL of library is equal to 7 nM. Example calculation is below. Adjust dilution factor accordingly. 2 uL of library+198 uL qubit solution:

$$\frac{\text{?} reading ( \frac{ng}{mL} )}{1 , 000} \times dilution factor ( 100 ) \times conversion factor ( 7 ) \text{?}$$

$$\text{?} \text{indicates text missing or illegible when filed}$$

### Example 5. Protocols for Normalization and Pooling

1. Normalize libraries to 5 nM: Dilute an aliquot (i.e. 4 μL) of each sample library to 5 nM using nuclease-free water or 10 mM Tris-Cl with 0.1% Tween-20, pH 8.5. An example calculation is as follows:

$$\frac{Library concentration \text{?} library}{\text{?}} \text{?} final volume of library$$

$$Final volume of library - 4 \text{?} library \text{?}$$

$$\text{?} \text{indicates text missing or illegible when filed}$$

2. Mix and spin: Mix the 5 nM libraries thoroughly by vortexing followed by spinning briefly.
3. Prepare library mix: Label a new 1.5 mL microtube for the library mix. Prepare a 5 nM mixture of libraries by combining each library at equal volume (i.e. mixing 5 μL of each 5 nM library). Gently pipette the entire solution up and down 10 times to mix thoroughly. The mixture can also be quickly vortexed and spun.
4. Quantify library pool (recommended): It is recommended that the library mix be quantitated using Qubit or another library quantitation method (qPCR) to ensure the mix is at 5 nM (±10%) to prevent over- or under-clustering on the MiSeq. If the final dilution is not 5 nM (±10%), adjust the dilution for loading the sequencer accordingly to obtain the desired concentration.

### Example 6. Protocols for Preparing Diluted Libraries for Sequencing

Samples can be multiplexed and sequenced on the MiSeq using the v3 chemistry or the NextSeq. The number of samples that can be loaded is dependent on the number of paired-end reads per sample and sequencing depths that required.

The maximum number of samples that can be loaded on each kit is displayed in a table. Choose the appropriate sequencing workflow and kit based on the number of samples to be sequenced.

### Sequencing on MiSeq (MiSeq v3 Kit)

For v3 chemistry (MiSeq v3 kit), dilute libraries to 5 nM. The final concentration of the libraries for sequencing is 20 pM.

1. Prepare 0.2 N NaOH: Label a new 1.5 mL microtube for 0.2 N NaOH. Prepare the NaOH by combining 800 μL nuclease-free water with 200 μL of 1 N NaOH. Vortex the solution to mix.
Alternately, prepare a 1 N NaOH solution by combining 500 μL 10 N NaOH into 4.5 mL of nuclease-free water. Vortex the solution to mix. If 1 N NaOH has not been prepared within the last week from a 10 N solution, prepare a new 1 N NaOH solution.

2. Denature the library mix: Label a new 1.5 mL microtube for the denatured, 20 pM library mix.
a. Denature the library mix by combining 5 μL of the library mix and 5 μL of the freshly prepared 0.2 N NaOH.
b. Vortex the solution thoroughly for 10 seconds and centrifuge the solution in a microfuge for 1 minute.
c. Let the solution stand at room temperature for 5 minutes.
d. Add 990 μL of Illumina's HT1 solution to the denatured library mix.
e. Invert the mixture several times, spin briefly, and place on ice.

3. Dilute to 20 pM library mix: Label a new 1.5 mL microtube for the 20 pM library mix. Combine 480 μL of the 25 pM library mix (step 5) with 120 μL of Illumina's HT1 solution. Adjust the volumes as needed for libraries that are over or under 25 pM. Invert the mixture several times, spin briefly, and place on ice.

4. Combine library mix and PhiX control: Label a new 1.5 mL microtube for the mixture that will be loaded. Combine 594 μL of the 20 pM library mix (step 6) with 6 μL of a 20 pM PhiX library control. Briefly vortex, spin, and place on ice.

5. Load MiSeq cartridge: Using a clean 1000 μL tip, puncture the foil cap above the sample loading tube on the MiSeq cartridge. Load the 600 μL library mix and PhiX mixture (step 7) into the cartridge and ensure the solution has reached the bottom of the tube by lightly tapping the tube if liquid remains on the side wall or if there is an air bubble at the to bottom of the tube.

6. Run the MiSeq: Run the libraries on the MiSeq per the manufacturer's instructions using a paired-end read length of 75 (75) and two indexing reads of 8 cycles each: “MiSeq System User Guide”

7. Store diluted libraries and mixtures at −20° C. for long-term storage.

### Sequencing on the NextSeq

For sequencing on the NextSeq, dilute libraries to 5 nM. The final concentration of the libraries for sequencing is 1.8 pM.
1. Prepare 0.2 N NaOH: Label a new 1.5 mL microtube for 0.2 N NaOH. Prepare the NaOH by combining 800 μL nuclease-free water with 200 μL of 1 N NaOH. Vortex the solution to mix.

Alternately, prepare a 1 N NaOH solution by combining 500 μL 10 N NaOH into 4.5 mL of nuclease-free water. Vortex the solution to mix. If 1 N NaOH has not been prepared within the last week from a 10 N solution, prepare a new 1 N NaOH solution.
2. Denature the library mix: Label a new microtube for the denatured, 25 pM library mix.
   a. Denature the library mix by combining 5 μL of the library mix and 5 μL of the freshly prepared 0.2 N NaOH.
   b. Vortex the solution thoroughly for 10 seconds and centrifuge the solution in a microfuge for 1 minute.
   c. Let the solution stand at room temperature for 5 minutes.
   d. Add 5 μL of 200 mM Tris-HCl, pH 7.0.
   e. Vortex briefly and centrifuge the solution in a microfuge for 1 minute.
   f. Add 985 μL of Illumina's HT1 solution to the denatured library mix.
   g. Vortex briefly and centrifuge the solution in a microfuge for 1 minute.
3. Dilute 25 pM library mix to 1.8 pM: Dilute the denatured library to 1400 μL of a 1.8 pM solution by combining 101 μL of the 25 pM denatured library mix with 1299 μL of Illumina's HT1 solution. Invert to mix and spin briefly.
4. Combine library mix and PhiX control: Label a new 1.5 mL microtube for the mixture that will be loaded. Combine 1287 μL of the 1.8 pM library mix (step 3) with 13 μL of a 1.8 pM PhiX library control. Briefly vortex, spin, and place on ice.
5. Load NextSeq cartridge: Using a clean 1000 μL tip, puncture the foil cap above the sample loading well on the NextSeq cartridge. Load 1300 μL library mix and PhiX mixture (step 4) into the cartridge and ensure the solution has reached the bottom of the cartridge well.
6. Run the NextSeq: Run the libraries on the NextSeq per the manufacturer's instructions using a paired-end read length of 75 (2×75) and two indexing reads of 8 cycles each: “NextSeq System User Guide”.
7. Store Libraries: Store diluted libraries and mixtures at −20° C. for long-term storage.

### Example 7. Detecting Somatic Mutations in ctDNA Reference Material

Seraseq™ ctDNA Reference Material was obtained from SeraCare and was used to prepare cfDNA samples for testing in this example. Seraseq ctDNA Reference Material consists of DNA purified from a reference cell line, GM24385, plus constructs containing variants mixed at a defined allele frequency. Processing of the purified DNA produces an average DNA fragment size of approximately 170 base pairs. Somatic mutations present in Seraseq™ ctDNA Reference Material are in gene ID AKT1, APC, ATM, BRAF, CTNNB1, EGFR, ERBB2, FGFR3, FLT3, FOXL2, GNA11, GNAQ, GNAS, IDH1, JAK2, KIT, KRAS, MPL, NCOA4-RET, NPM1, NRAS/CSDE1, PDGFRA, PIK3CA, PTEN, RET, SMAD4, TP53, and TPR-ALK. Most of the targets in this Reference Material are not on the same gene and do not have overlapping regions, and therefore, SLIMAMP® tag is not used in the primer design.

21 genomic loci were selected to cover hot spots in common cancer related genes in the Seraseq™ ctDNA reference material by the Pillar PIVAT® software for the liquid biopsy monitoring panel.

A 21-plex customized panel of primer pairs targeting 21 specific genomic loci of the SeraCare reference material in a single multiplex reaction was designed by the Pillar AmpPD software, and manufactured at IDT. The target-specific sequences (without tag) of the 21 primer pairs and their target Gene ID are shown Table 7 below.

TABLE 7

| | Target-Specific | Target-Specific |
|---|---|---|
| | Forward Primer Sequence | Reverse Primer Sequence |
| Gene ID | SEQ ID NO | SEQ ID NO |
| MPL | GGCCTCAGCGCCGTCCT 1 | GCGGTACCTGTAGTGTGCA 2 |
| PTEN | AGTTCATGTACTTTGAGTTCCCTCA 3 | AGAACTCTACTTTGATATCACCACACA |
| | | 4 |
| ATM | GTCAAAGAAAATTTGATTGAATTGATGGCA | GTGACATGACCTACTTACTGTACC |
| | 5 | 6 |
| FLT3 | CTGACAACATAGTTGGAATCACTCA 7 | AGTGGTGAAGATATGTGACTTTGGA 8 |
| SMAD4 | AGGCGGCTACTGCACAAG 9 | TGGGCCAGGGATGTTTCCT 10 |
| GNAH | GTCCTTTCAGGATGGTGGATGT 11 | AGCAGTGGATCCACTTCCTC 12 |
| IDH1 | GCCAACATGACTTACTTGATCCC 13 | TGAGTGGATGGGTAAAACCTATCA 14 |
| GNAS | TTTCAGGACCTGCTTCGCT 15 | CCACCTGGAACTTGGTCTCAAA 16 |
| CTNNB1 | GGACTCTGGAATCCATTCTGGT 17 | CCTCAGGATTGCCTTTACCACT 18 |
| FOXL2 | GTAGTTGCCCTTCTCGAACATG 19 | CGCAAGGGCAACTACTGGA 20 |
| PIK3CA | CACGAGATCCTCTCTCTGAAATCA 21 | GCACTTACCTGTGACTCCATAGA 22 |
| PIK3CA | GGCTTTGGAGTATTTCATGAAACAAATG 23 | ATCCATTTTTGTTGTCCAGCCA 24 |
| PIK3CA | GATCTTCCACACAATTAAACAGCATG | GAGTGAGCTTTCATTTTCTCAGTTATCT |
| | 25 | 26 |
| FGFR3 | GGGTGGCCCCTGAGCGT 27 | AGCCCCGCCTGCAGGAT 28 |
| PDGFRA | TCGCTGGAGGGTCATTGAAT 29 | GCTGCATCGGGTCCACATAA 30 |
| PDGFRA | GAAAAATTGTGAAGATCTGTGACTTTGG 31 | GACACATAGTTCGAATCATGCATGA 32 |
| APC | CTCCACCACCTCCTCAAACA | GCAGTAGGTGCTTTATTTTTAGGTACTT |
| | 33 | 34 |
| EGFR | TCCAGGAAGCCTACGTGATG 35 | CCAGCAGGCGGCACACG 36 |
| EGFR | GCAGCATGTCAAGATCACAGATT 37 | TTCTTTCTCTTCCGCACCCA 38 |
| JAK2 | GCTTTCTCACAAGCATTTGGTTTT 39 | AGTTTTACTTACTCTCGTCTCCACA 40 |
| GNAQ | GTGTATCCATTTTCTTCTCTCTGACC 41 | AGTATTGTTAACCTTGCAGAATGGTC |
| | | 42 |

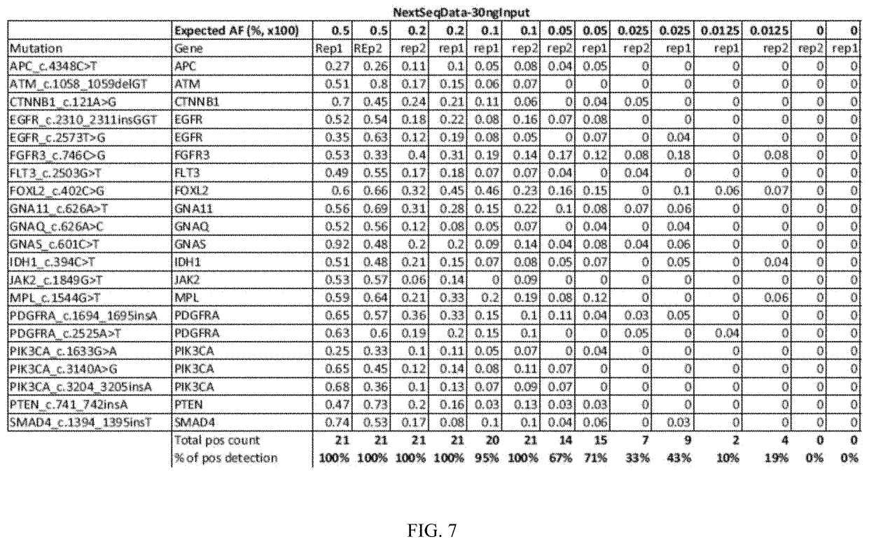

SERASEQ™ ctDNA Reference Material with 0.5% variant allele frequency was serial diluted 2.5, 5, 10, 20, and 40-fold in normal cfNDA from healthy donor to prepare cfDNA samples for testing; the expected allele frequencies (AF) are shown in FIG. 7 as 0.5, 0.2, 0.1, 0.05, 0.25, and 0.0125%, respectively. Each diluted sample was tested in duplicated.

All of 42 primers (21 pairs) were mixed into a single oligonucleotide pool and were added into a gene-specific PCR reaction with PCR polymerase, dNTP and buffer to amplify the 21 selected loci in 30 ng of DNA in each sample.

After Ampure beads purification, indexing PCR, a second Ampure beads purification, and quantitation and normalization, the libraries were sequenced on an Illumina NextSeq. The sequencing raw data were demuliplexed and converted to FASTQ by BCL2FASTQ, the subsequent FASTQ files were analyzed by the PIVAT® software. At least two out of 21 loci should be detected to call MRD-positive of a cfDNA sample. The results are shown in FIG. 7.

In this example, the detection sensitivity was as high as 0.0125% in expected AF, in which at least two loci were detected.

### Example 8. Designing Amplicon Panel Targeting Genomic Loci of Lung Panel

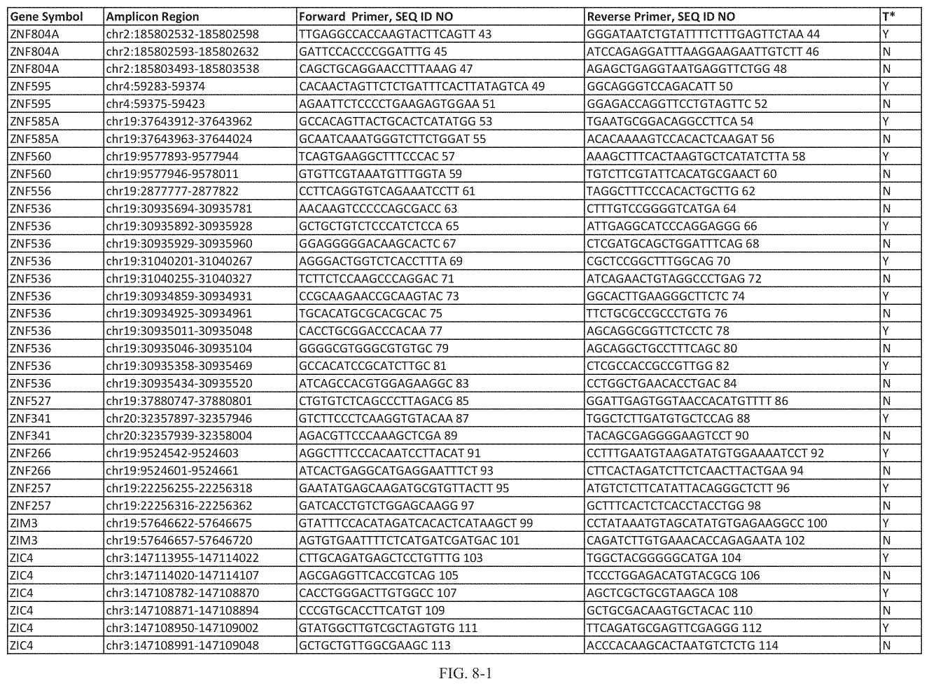

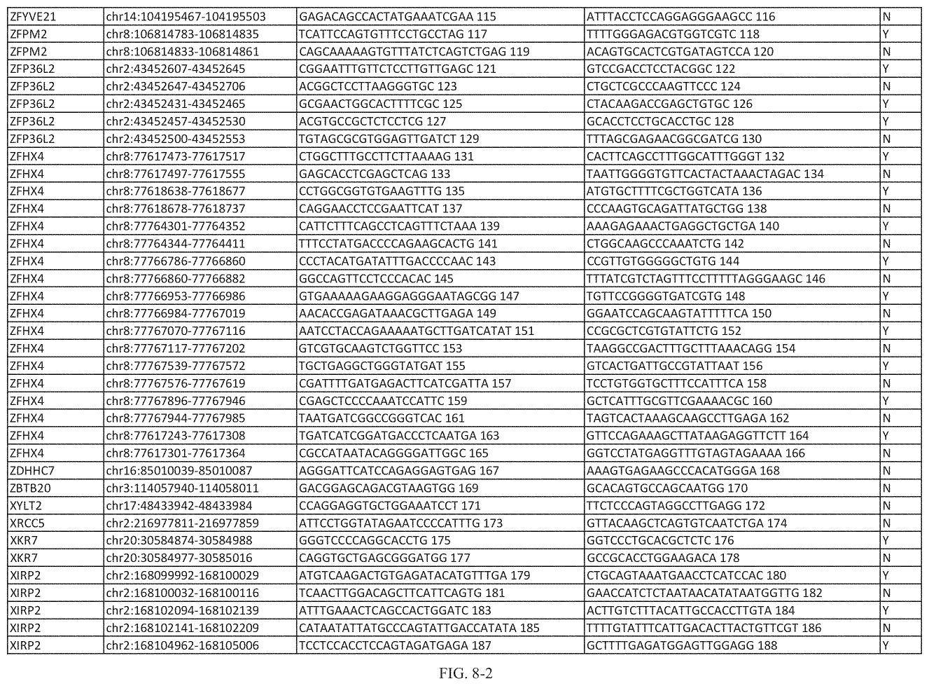

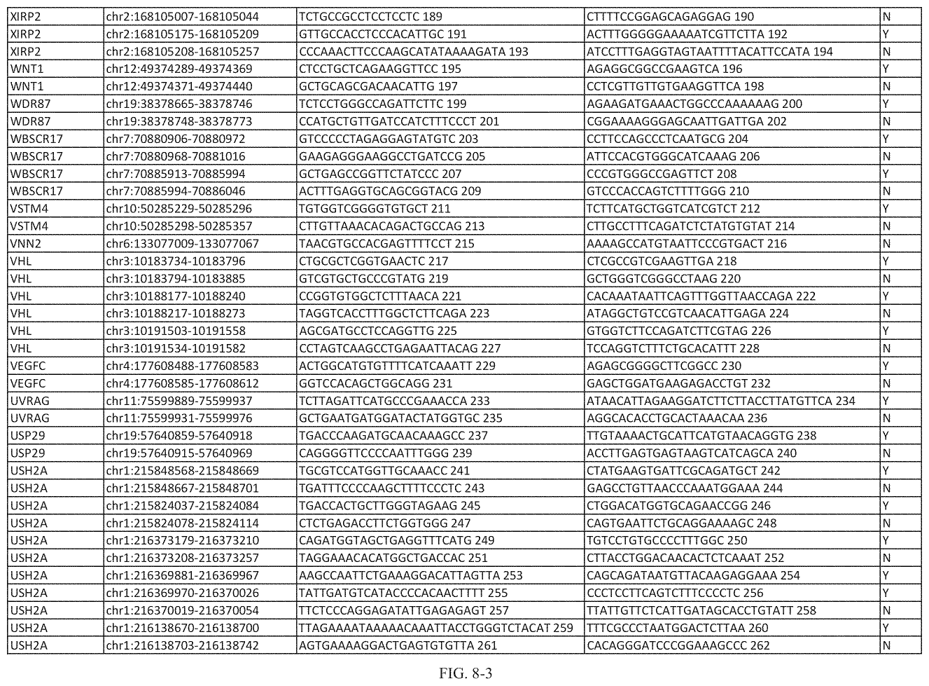

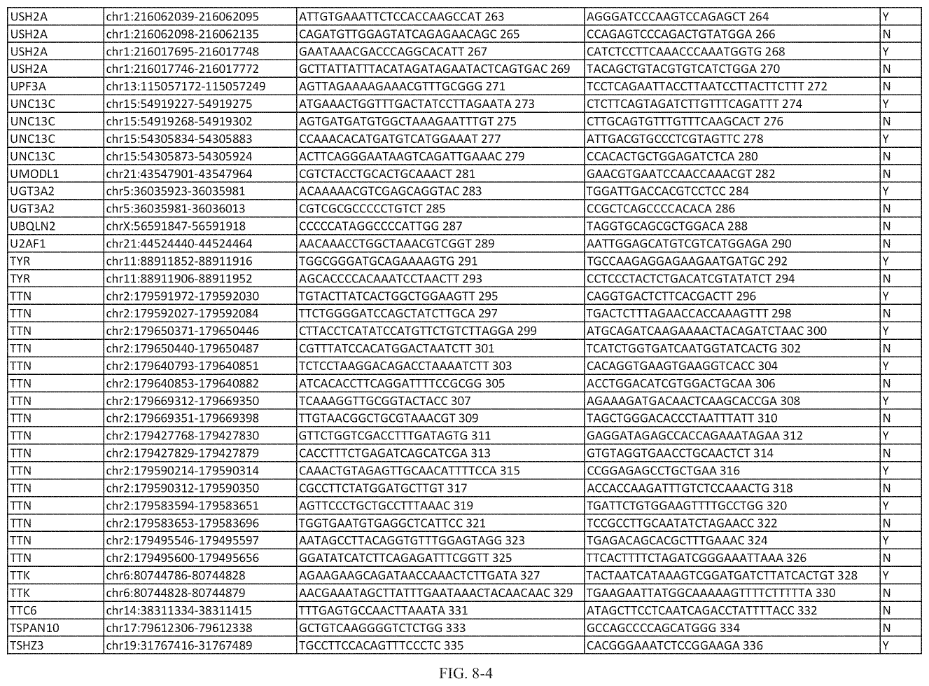

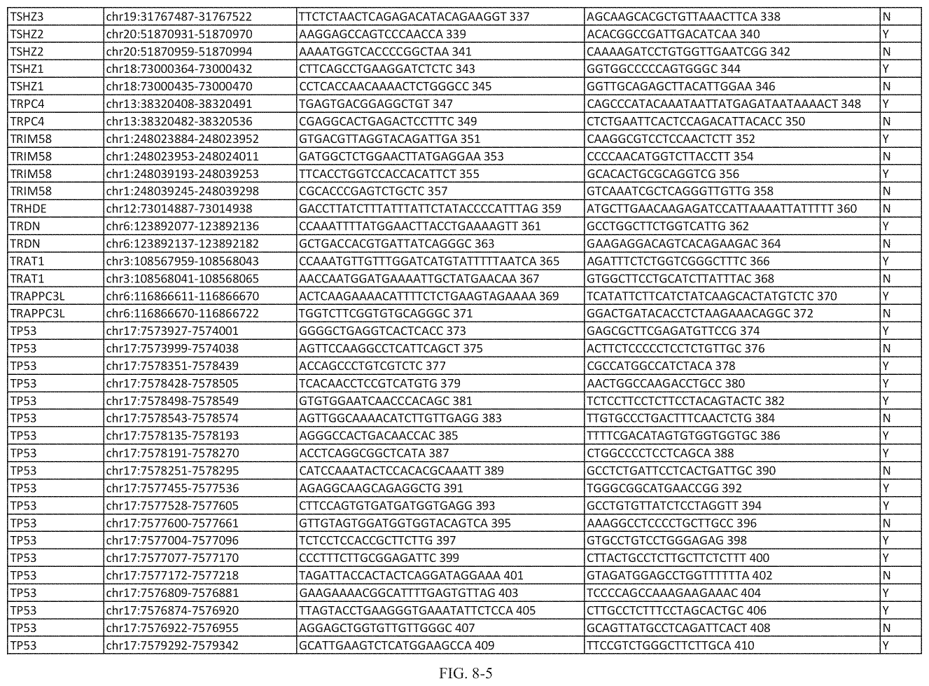

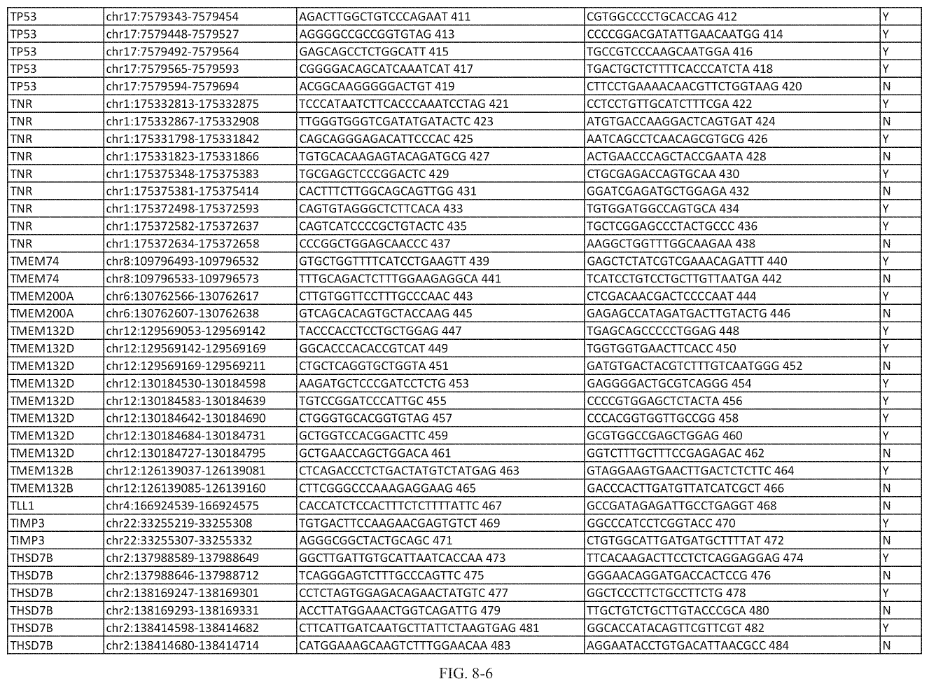

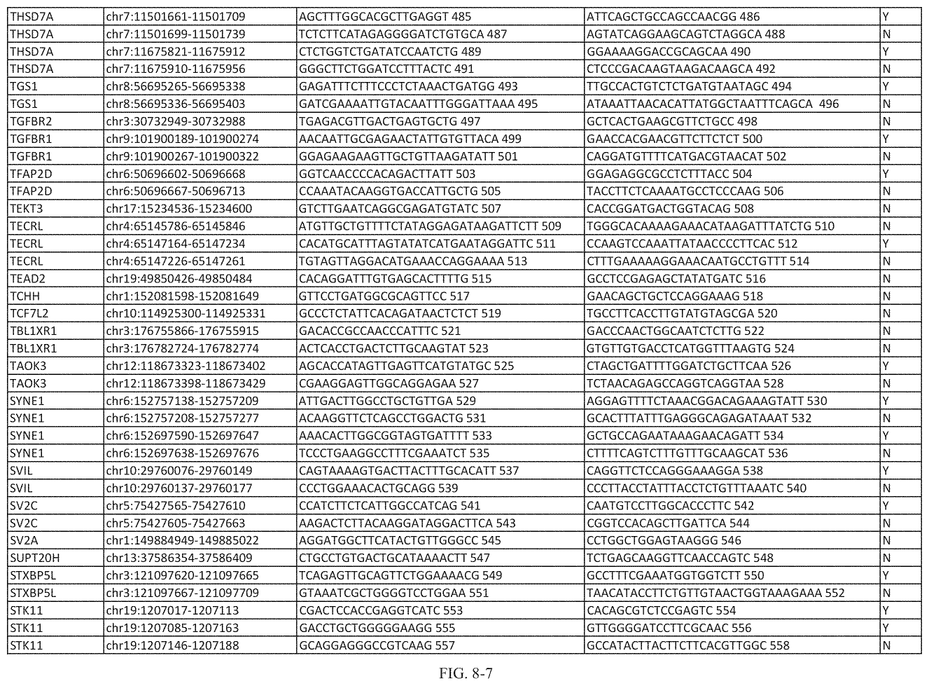

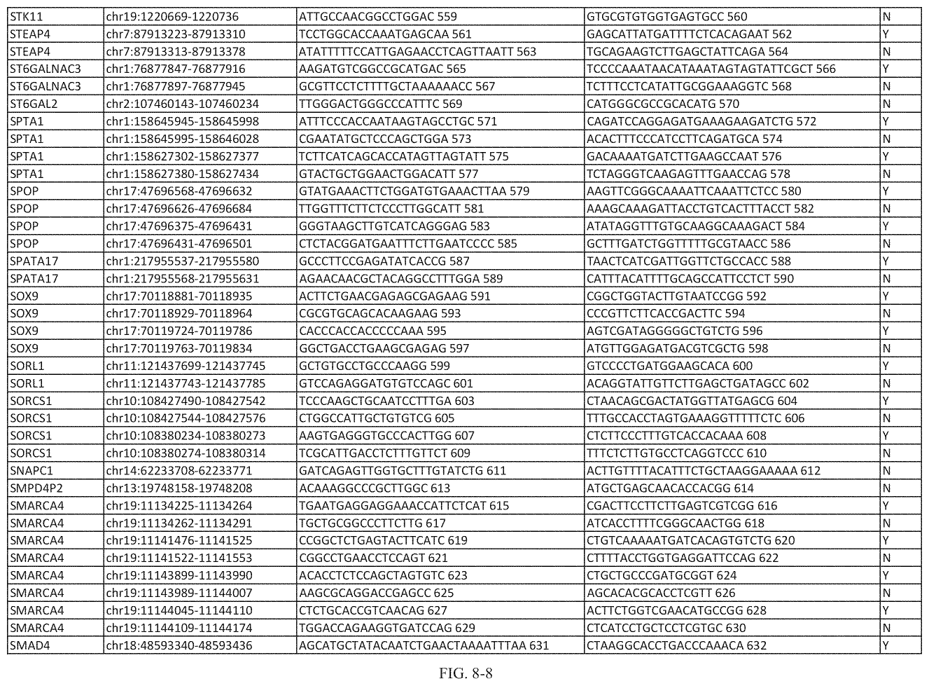

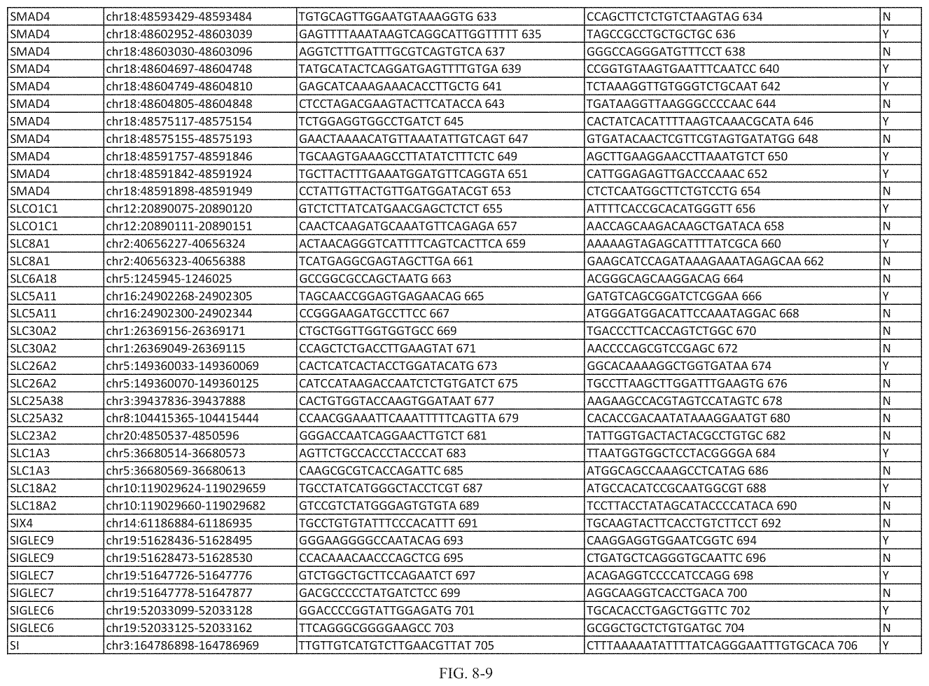

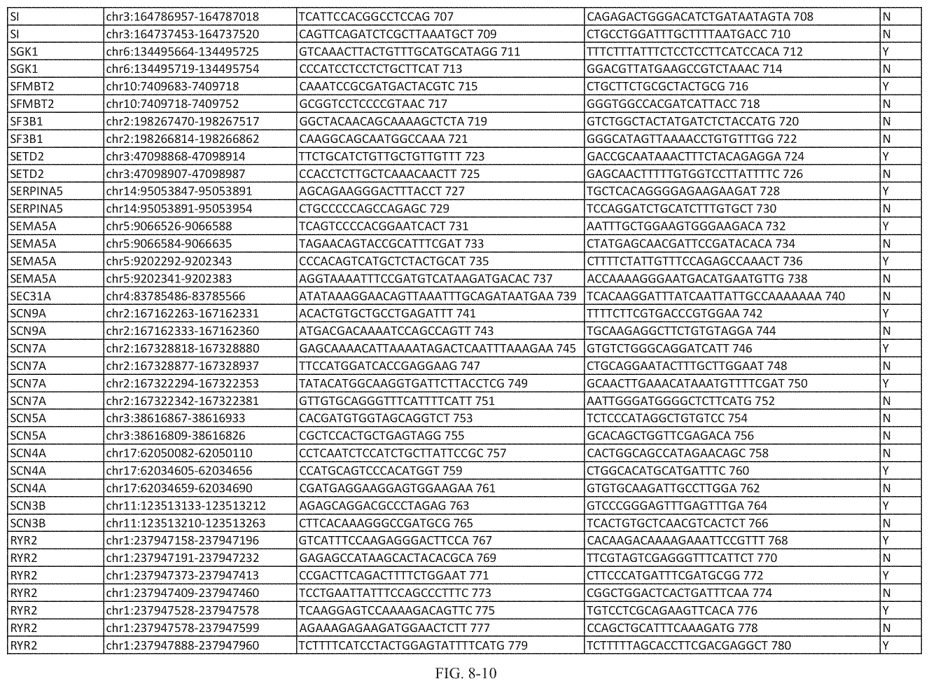

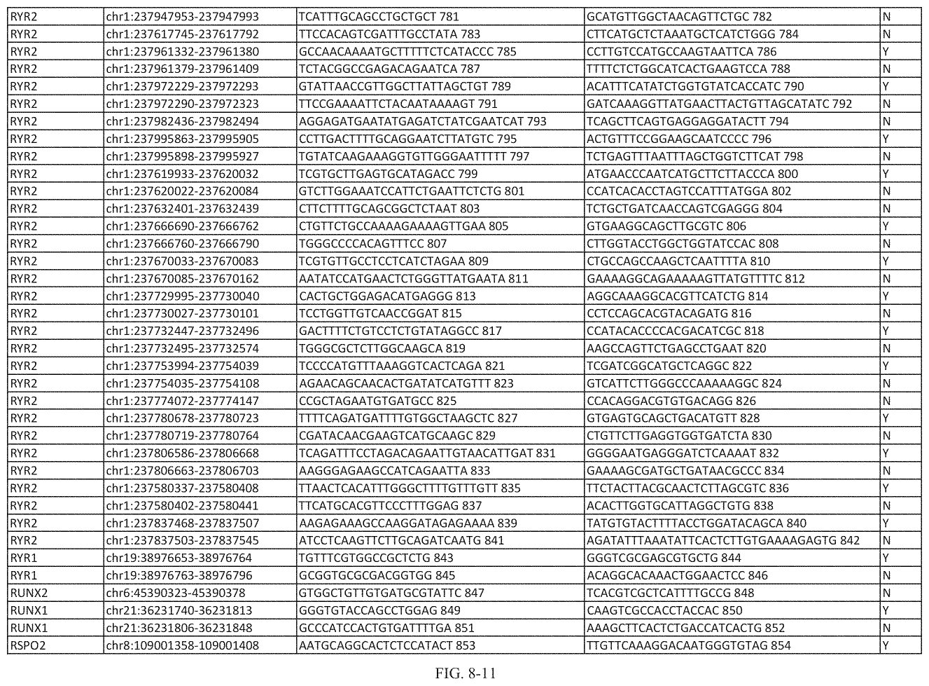

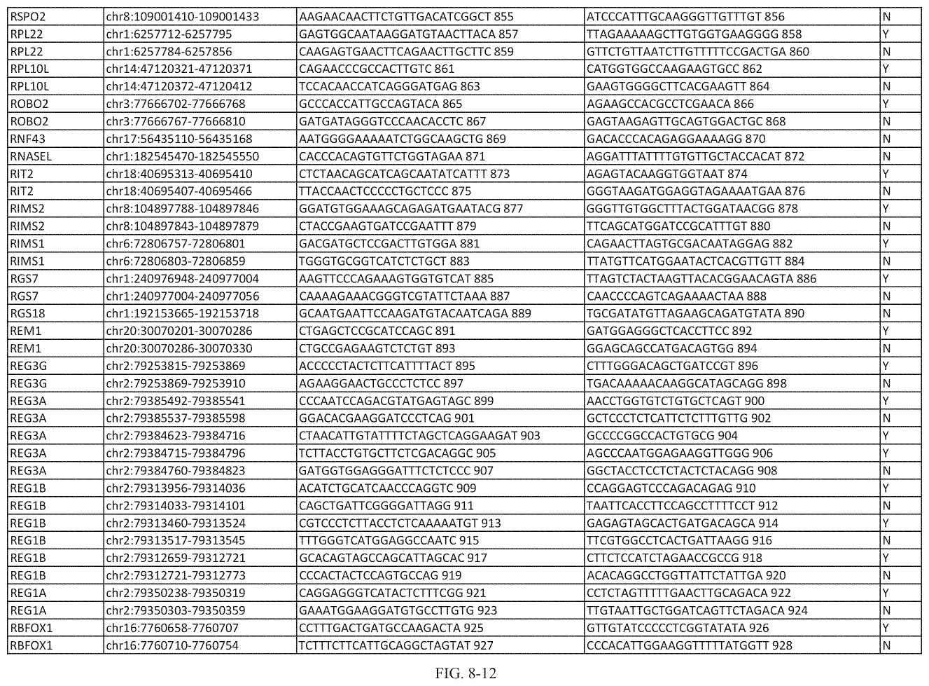

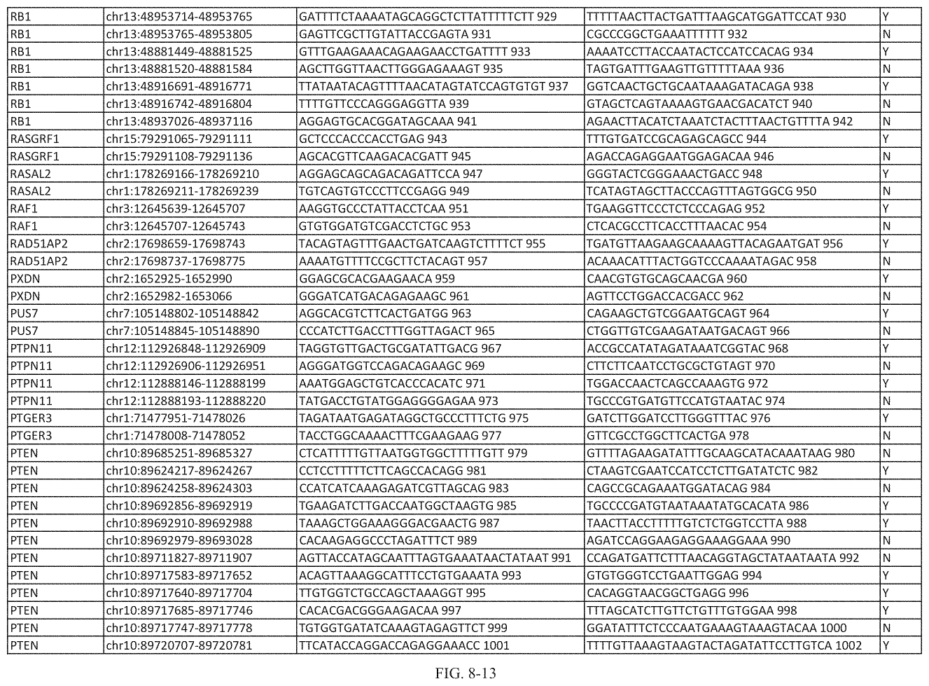

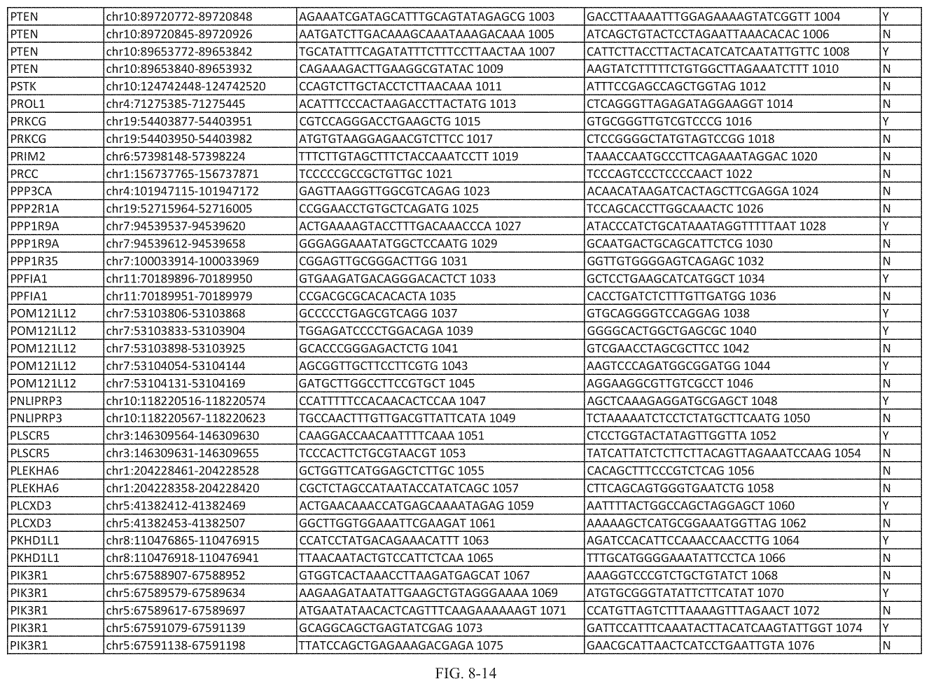

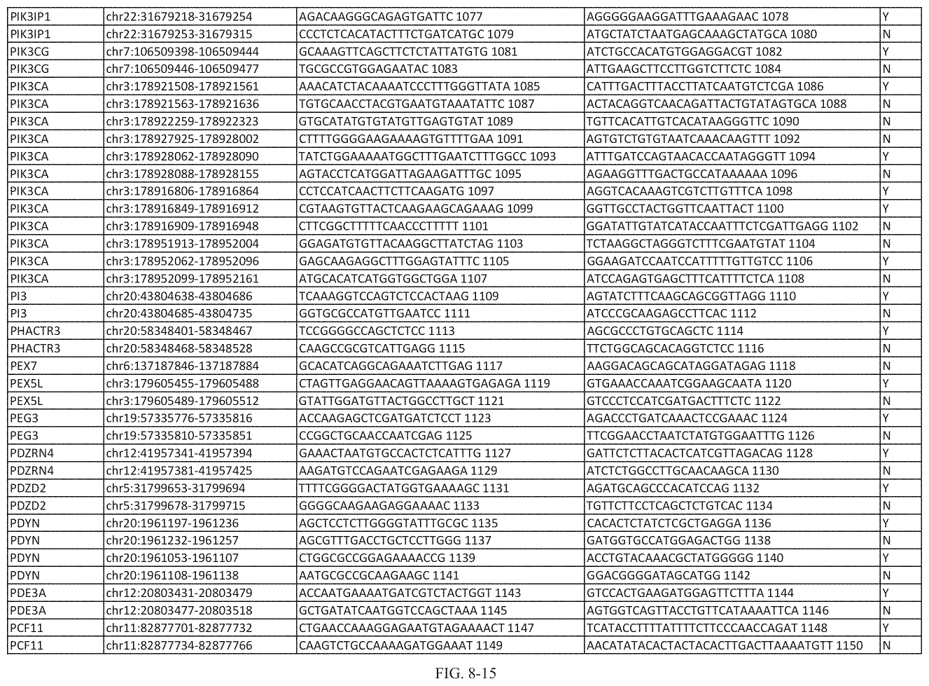

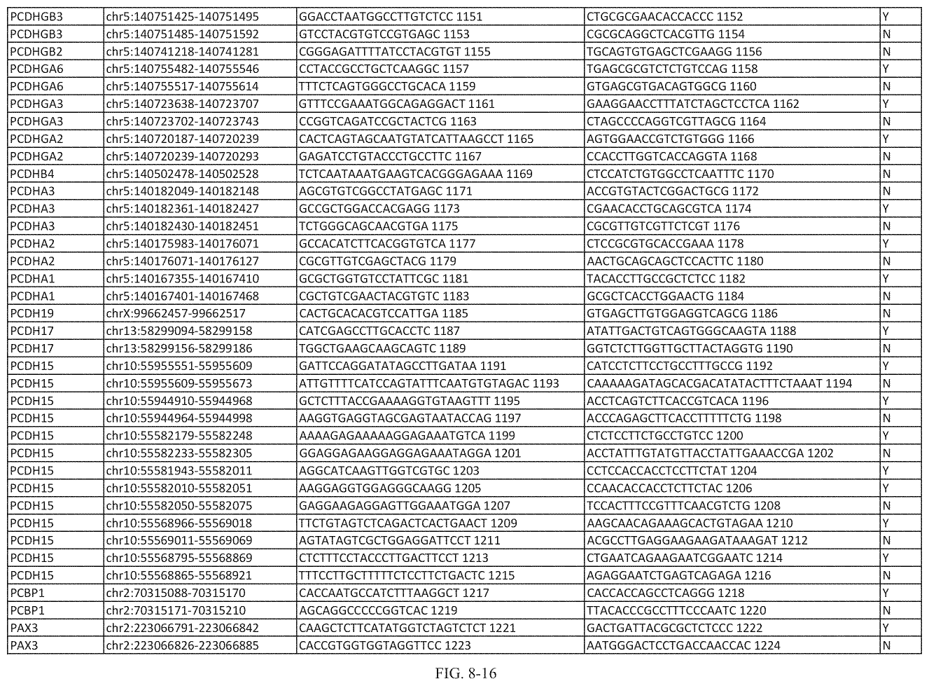

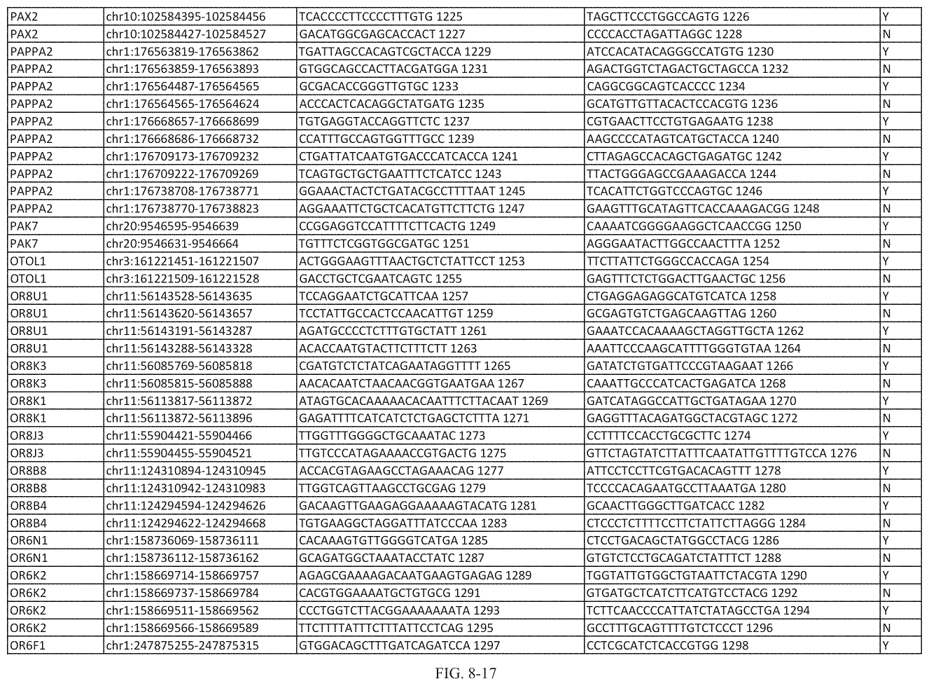

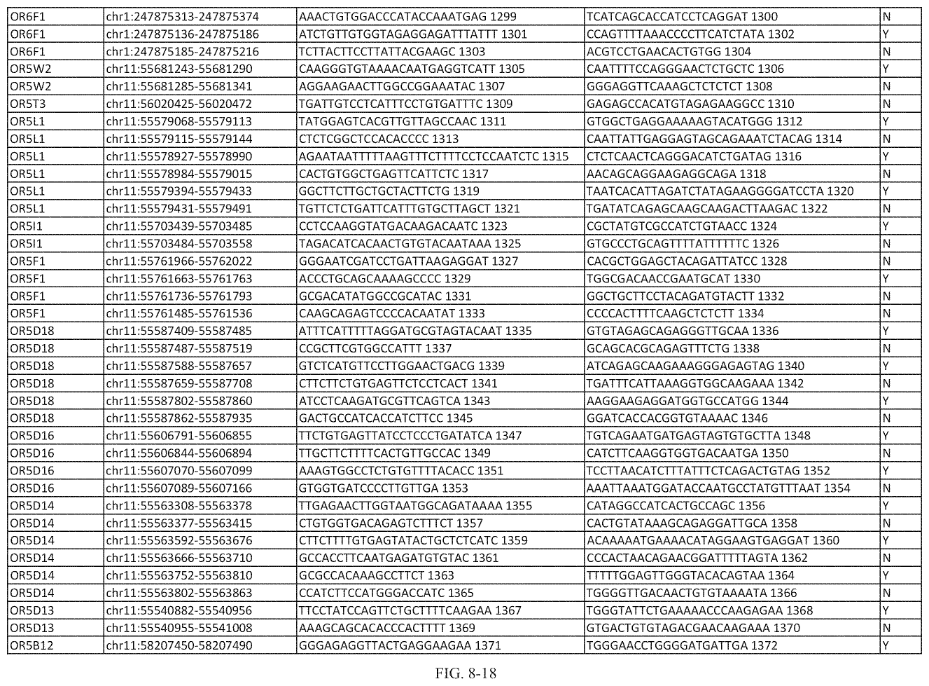

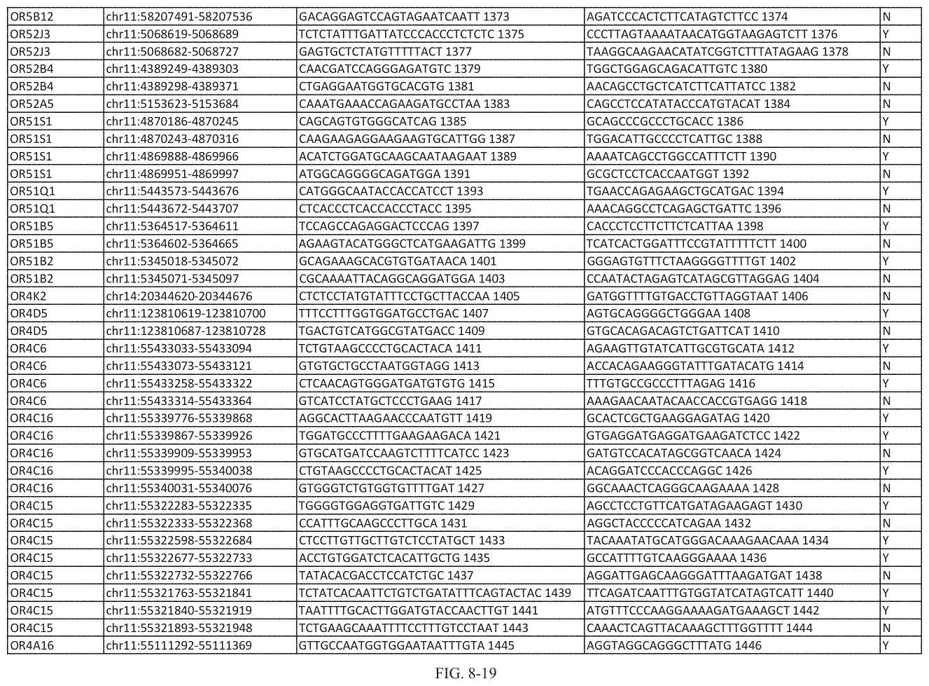

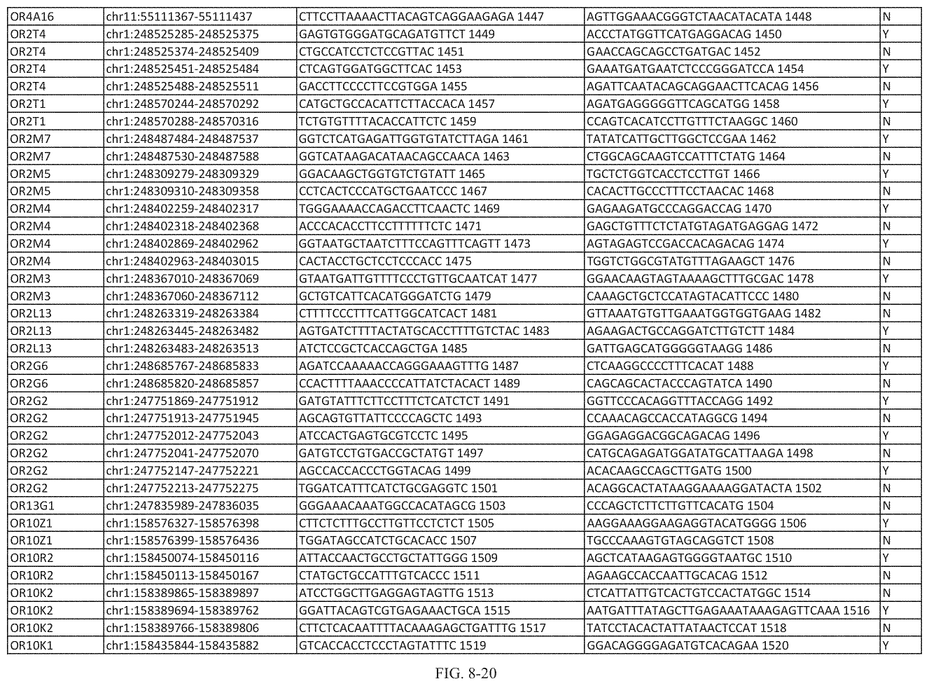

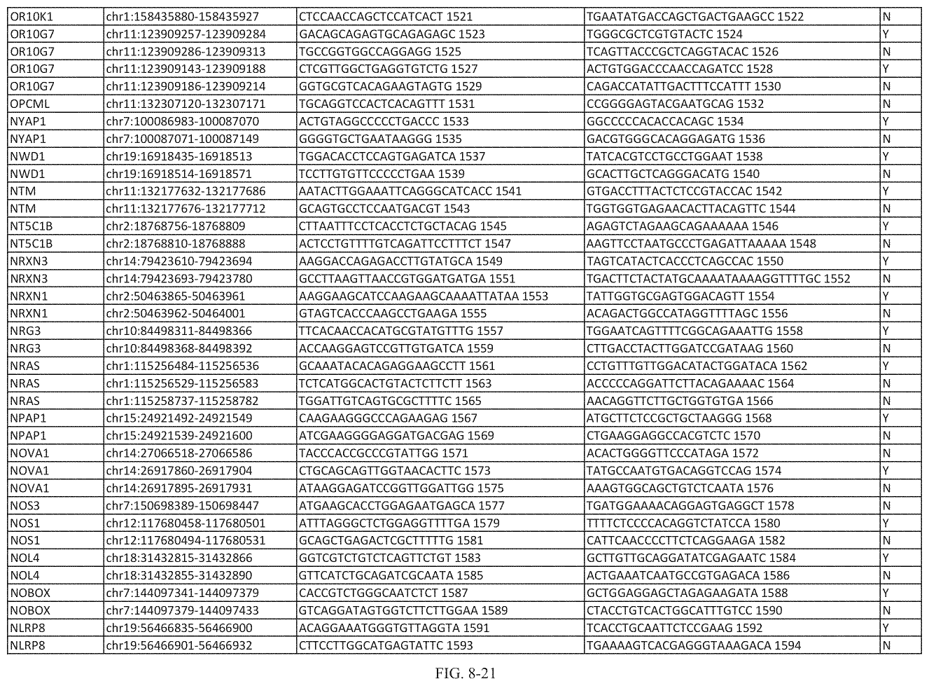

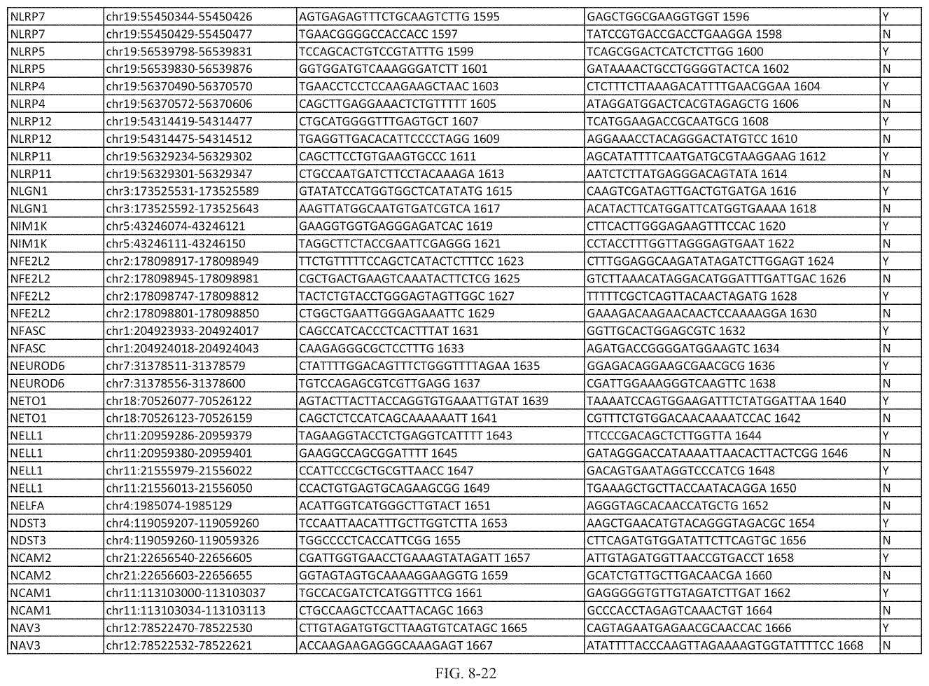

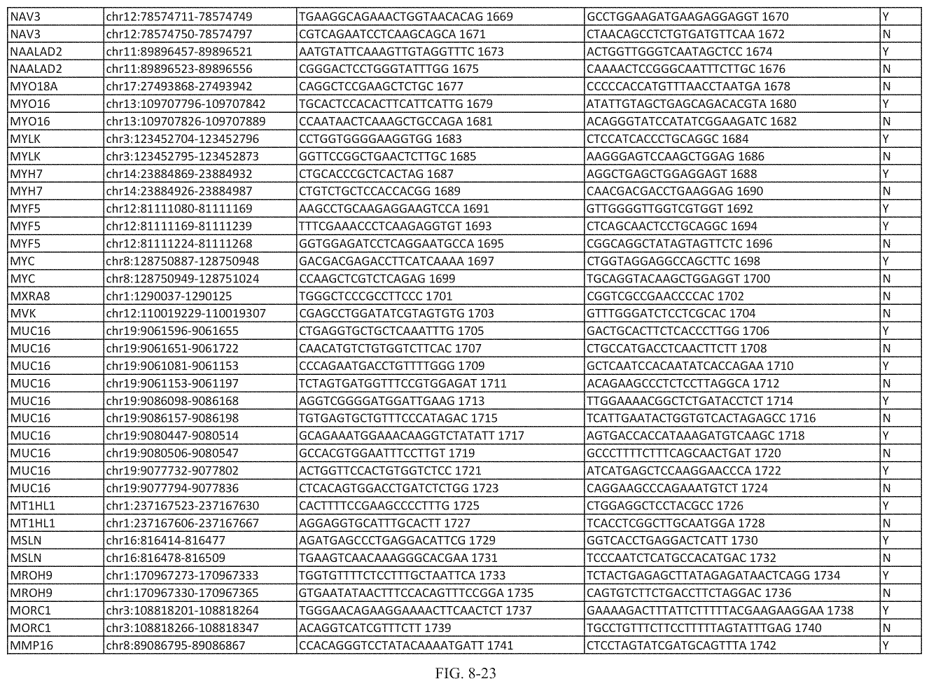

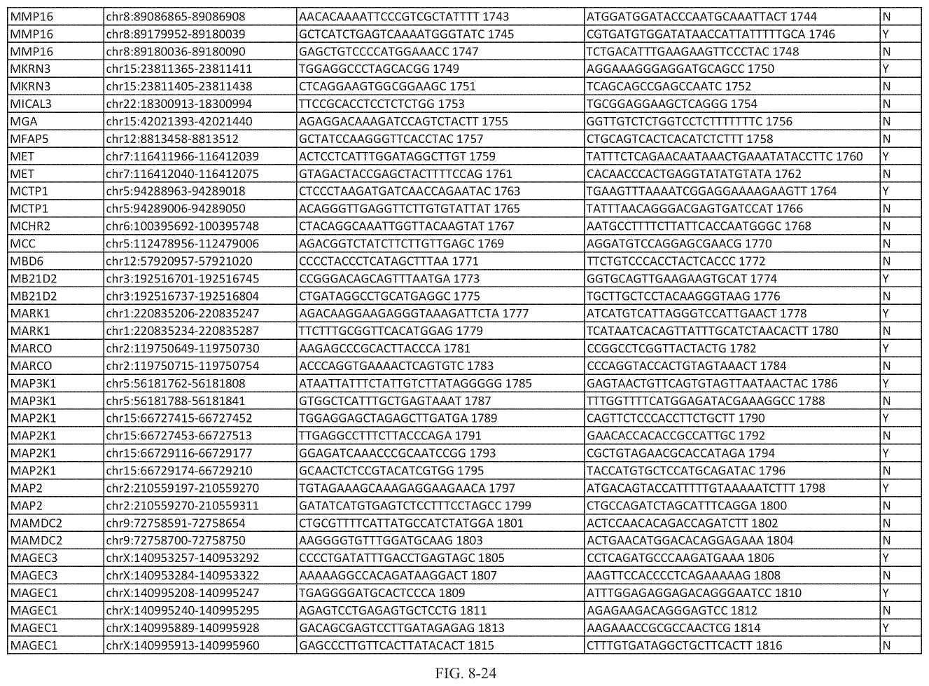

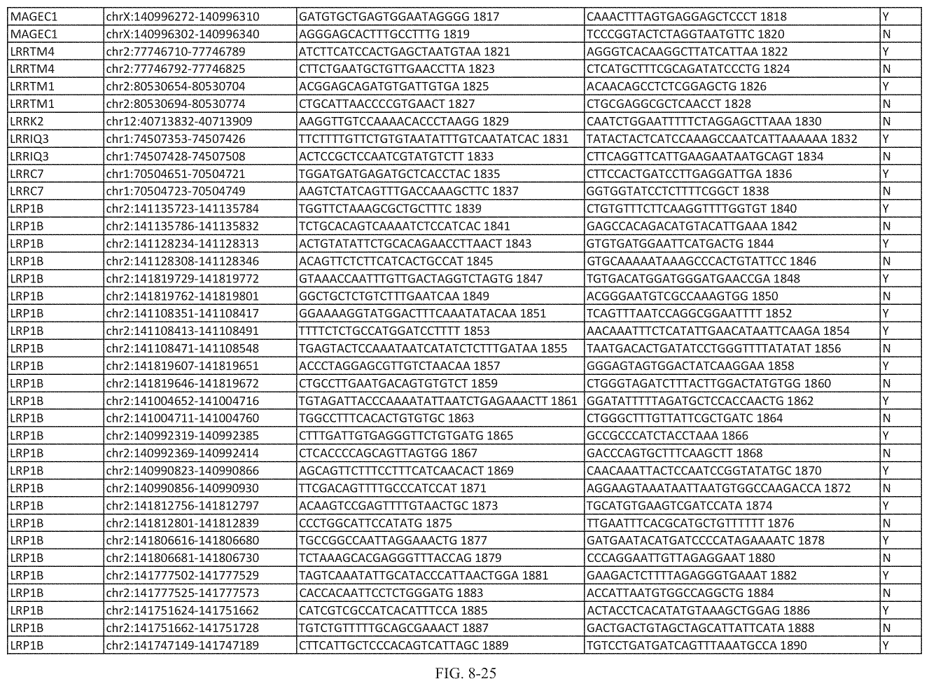

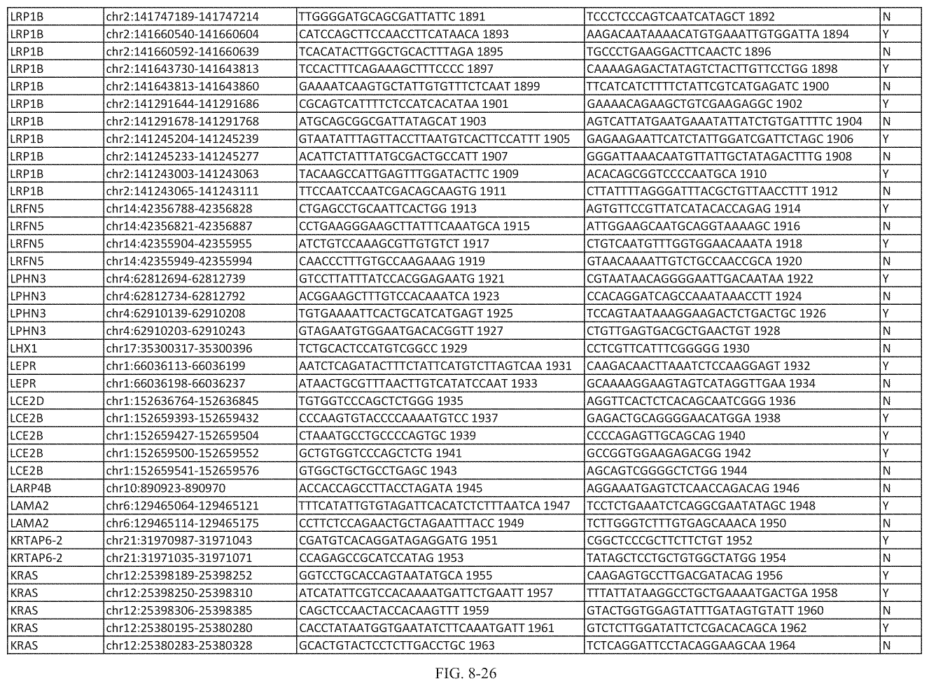

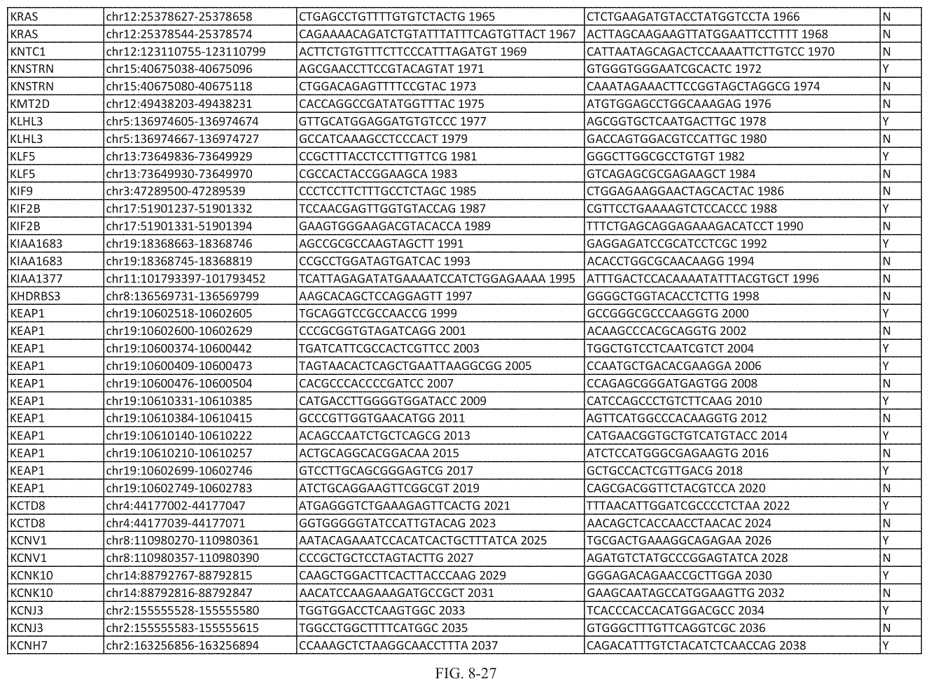

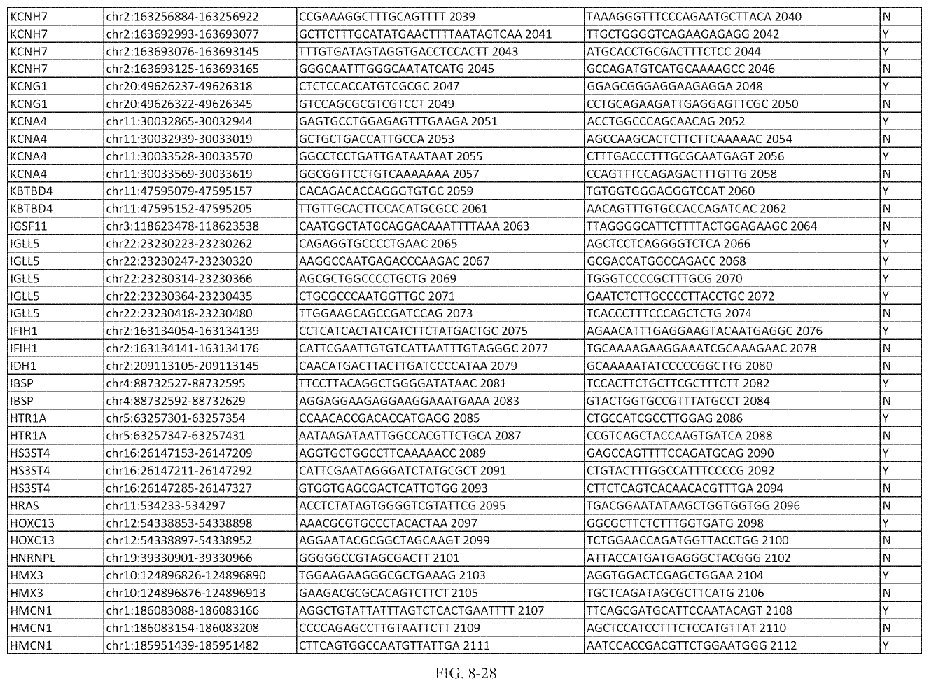

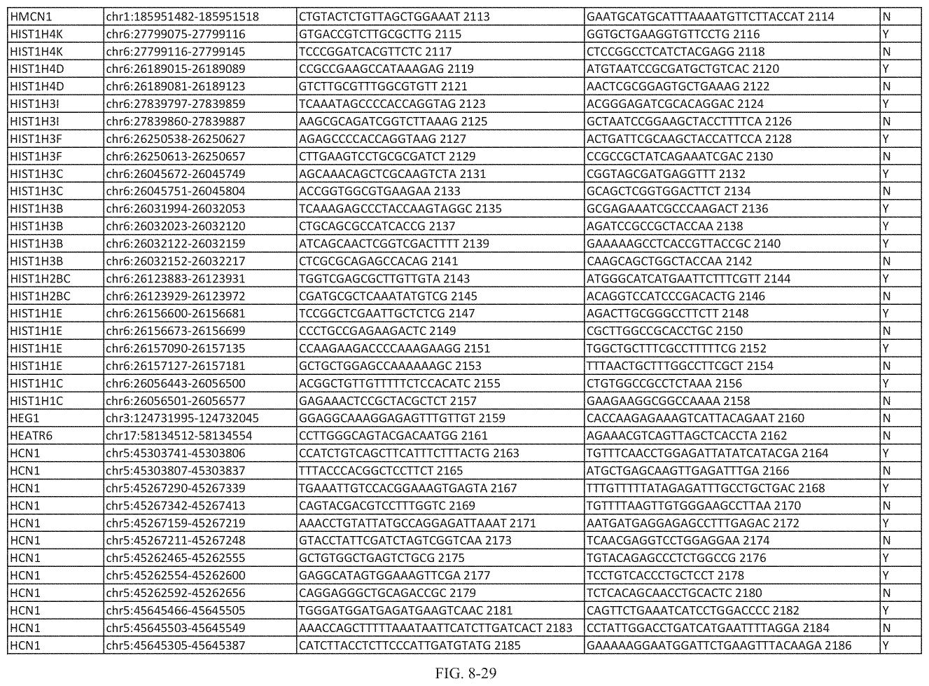

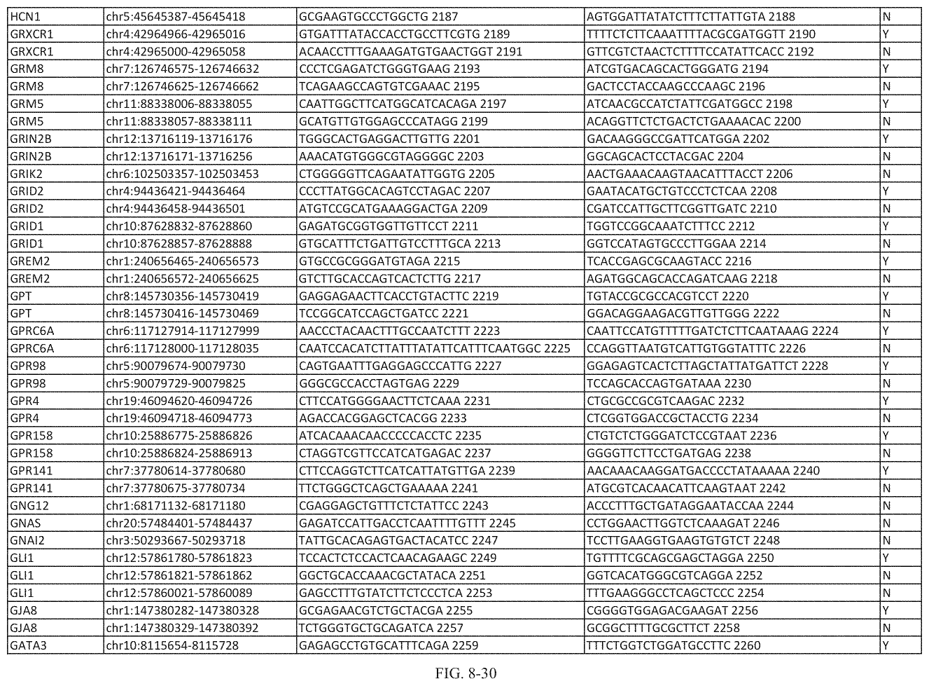

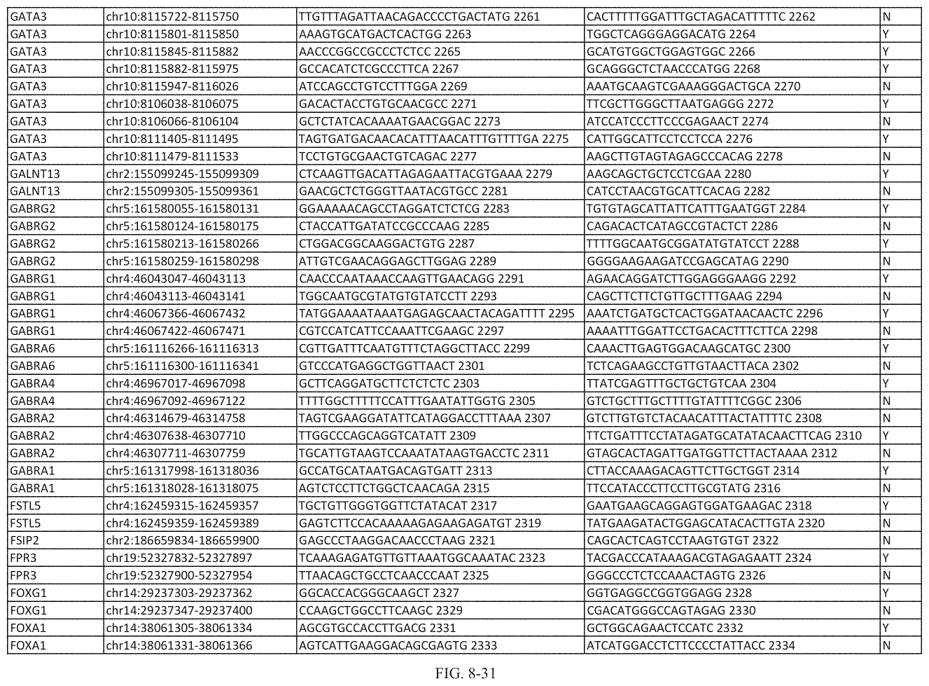

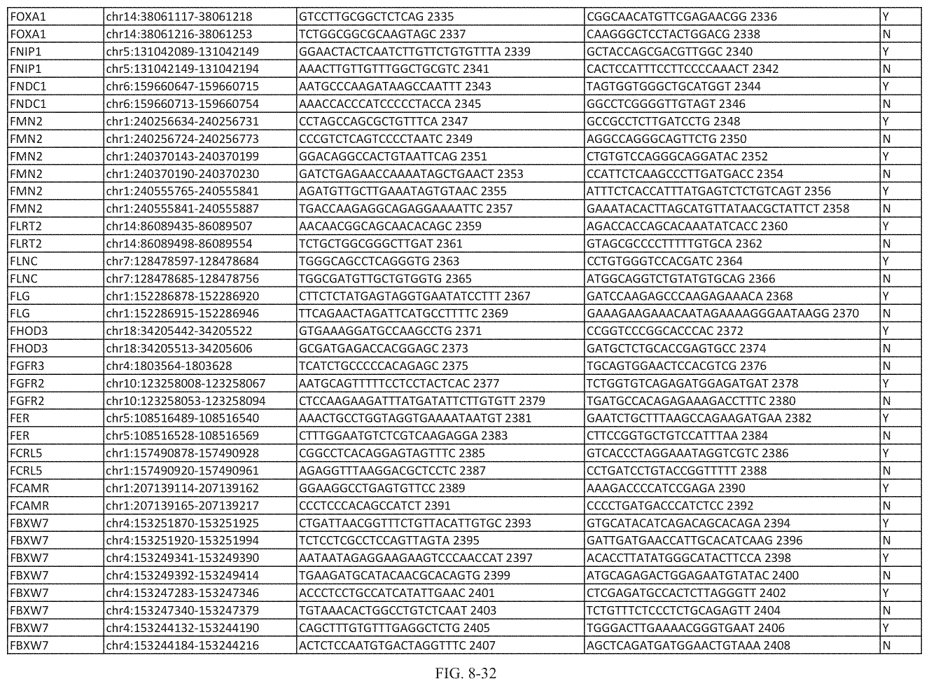

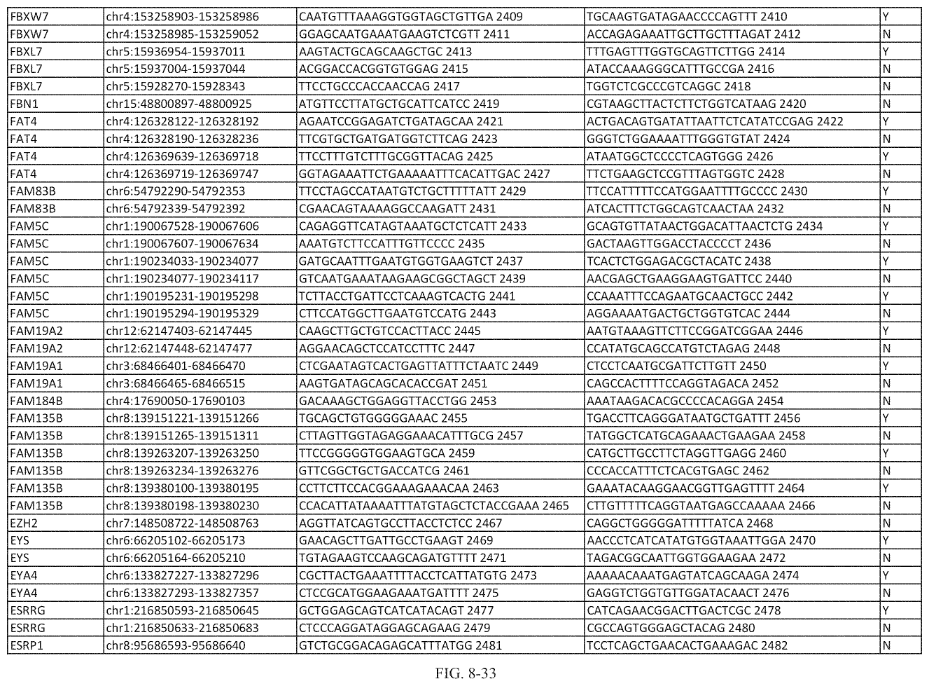

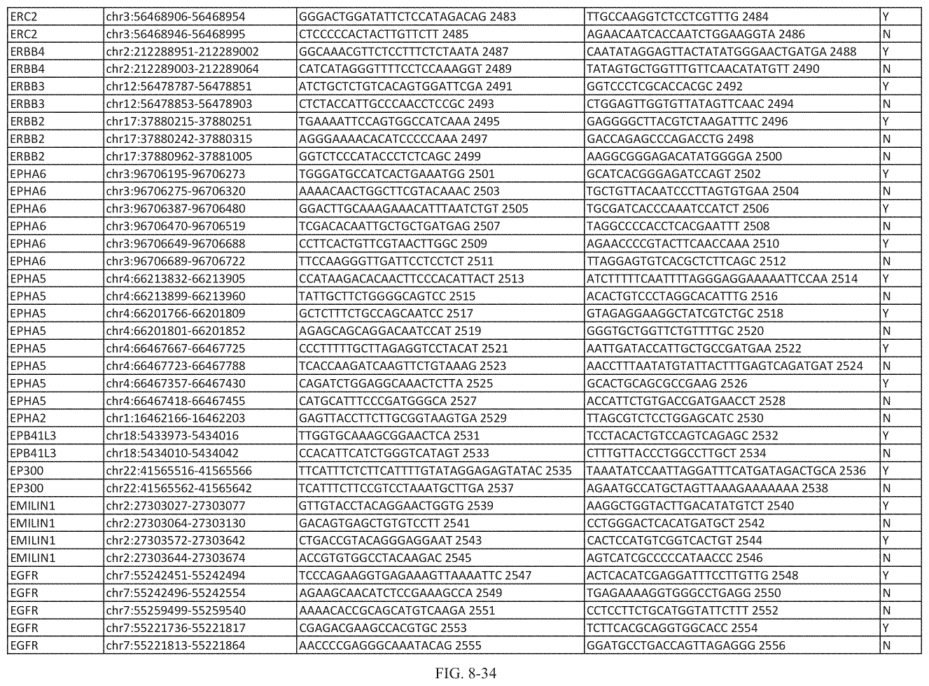

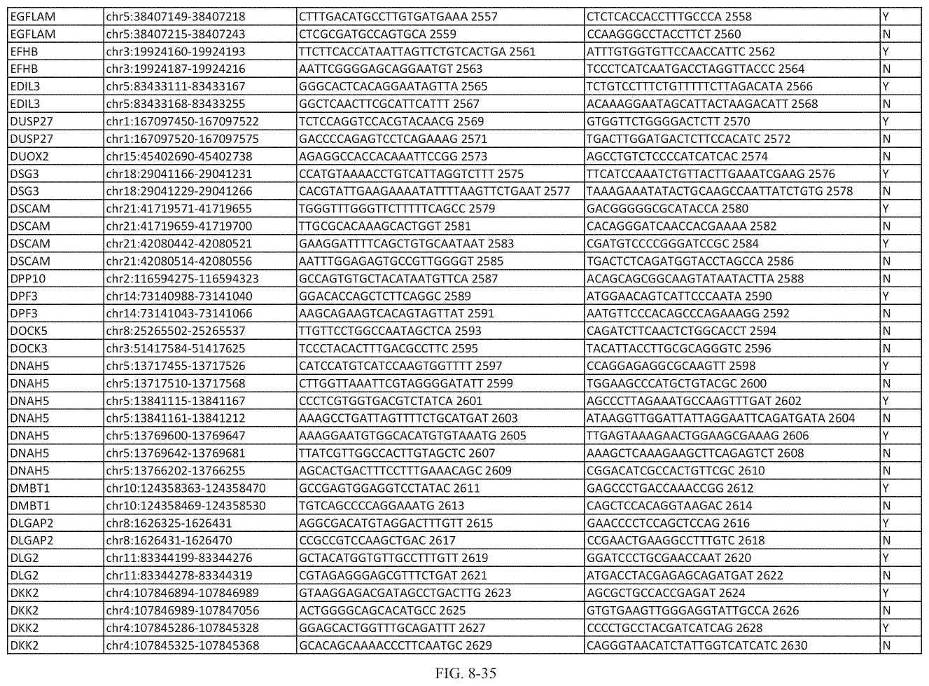

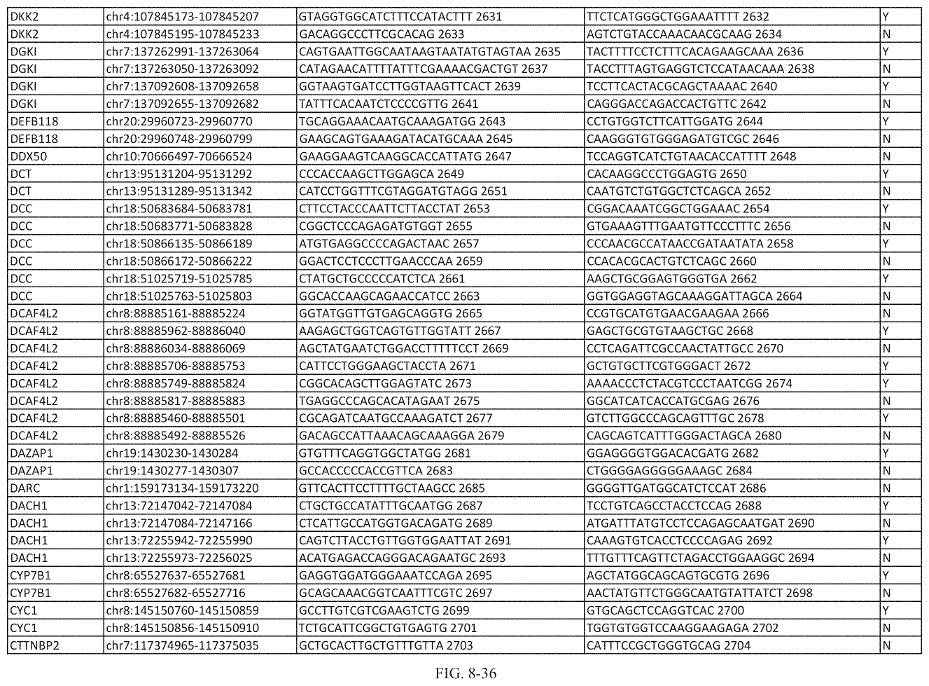

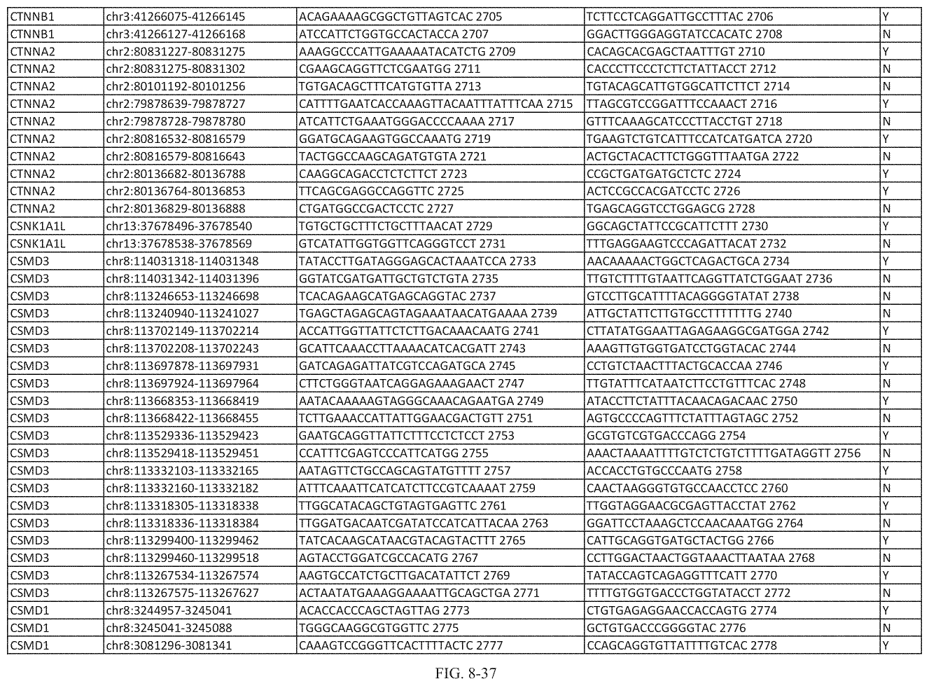

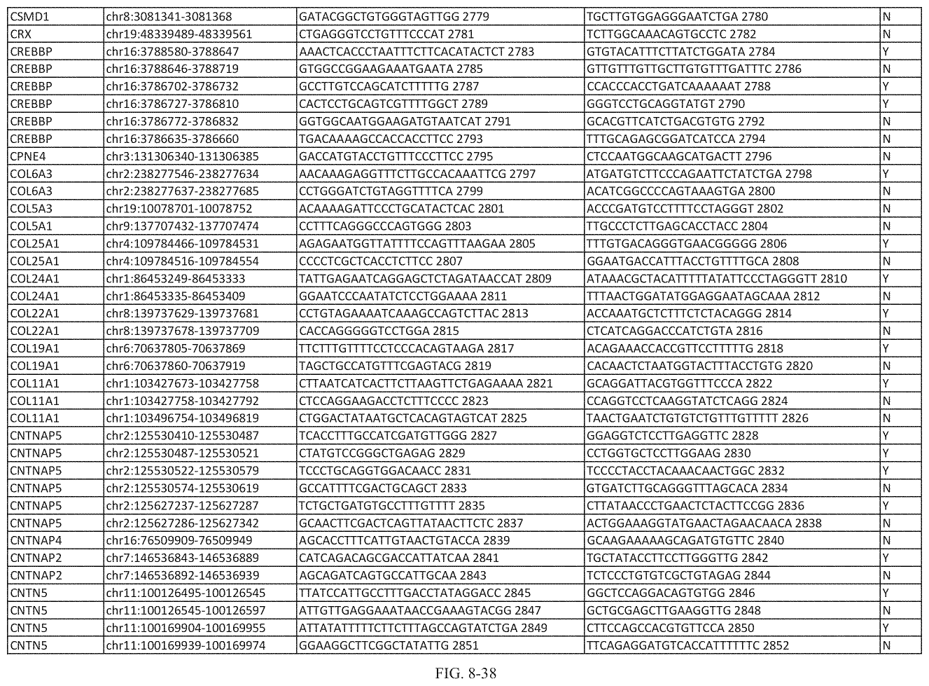

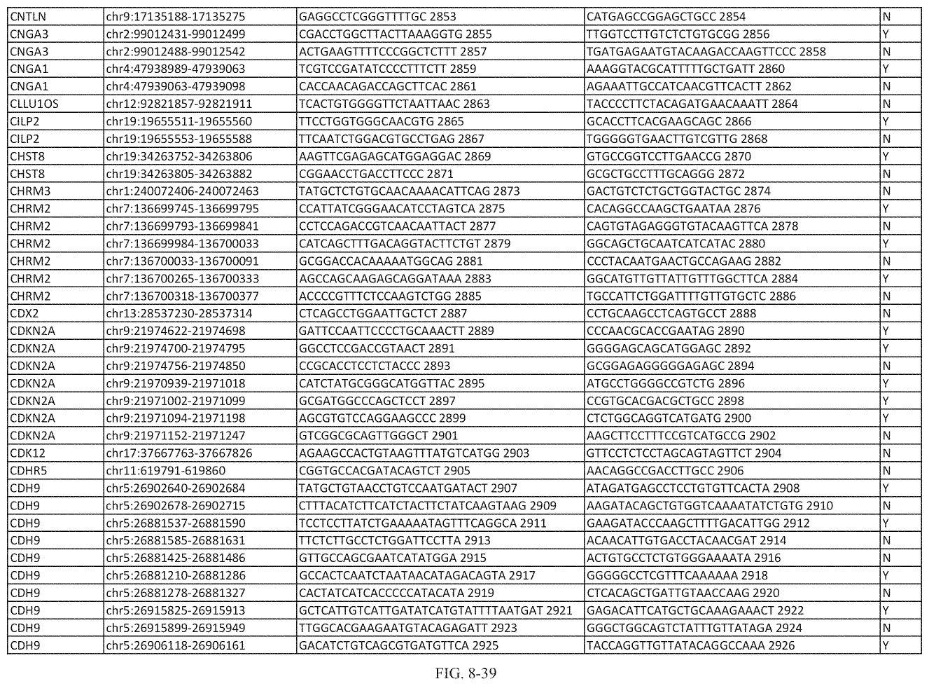

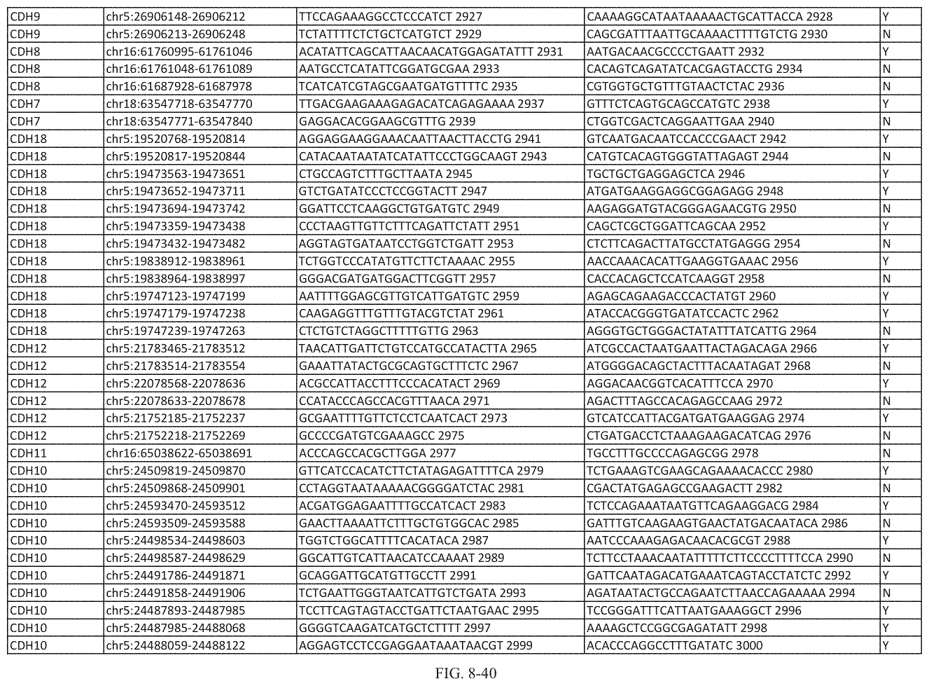

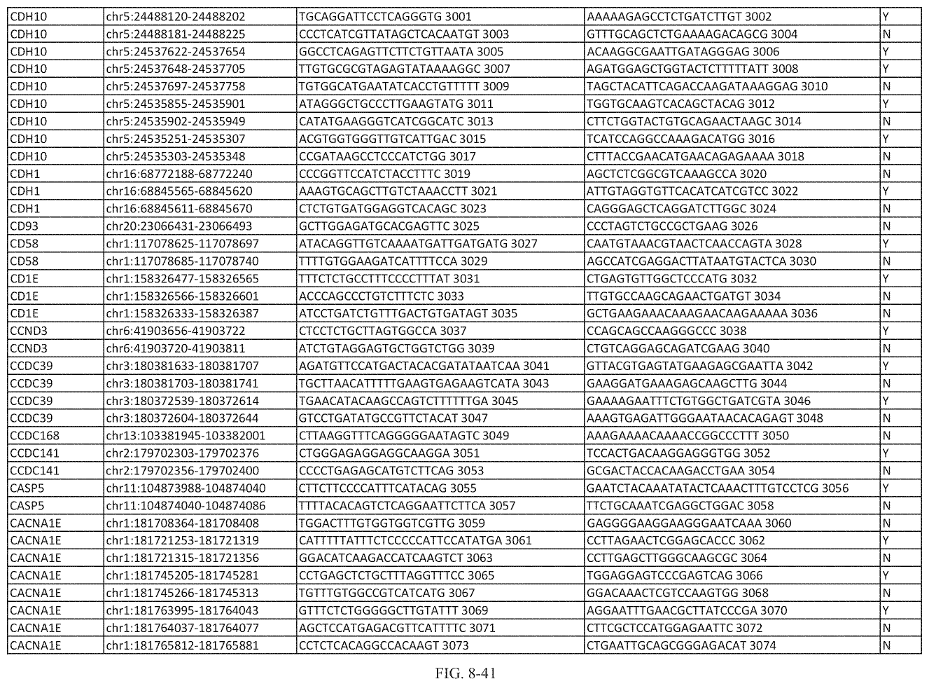

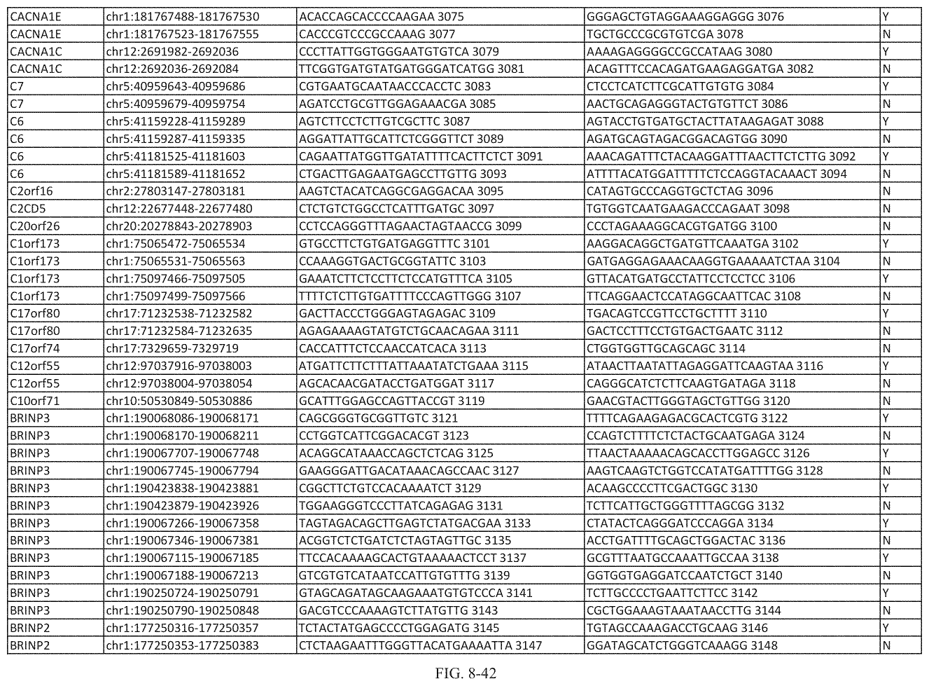

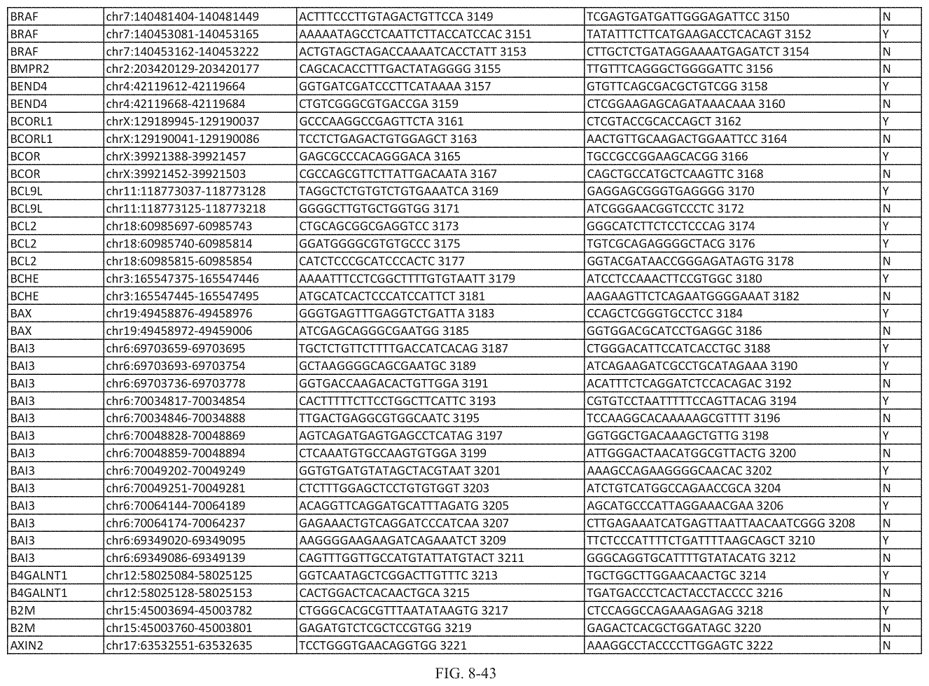

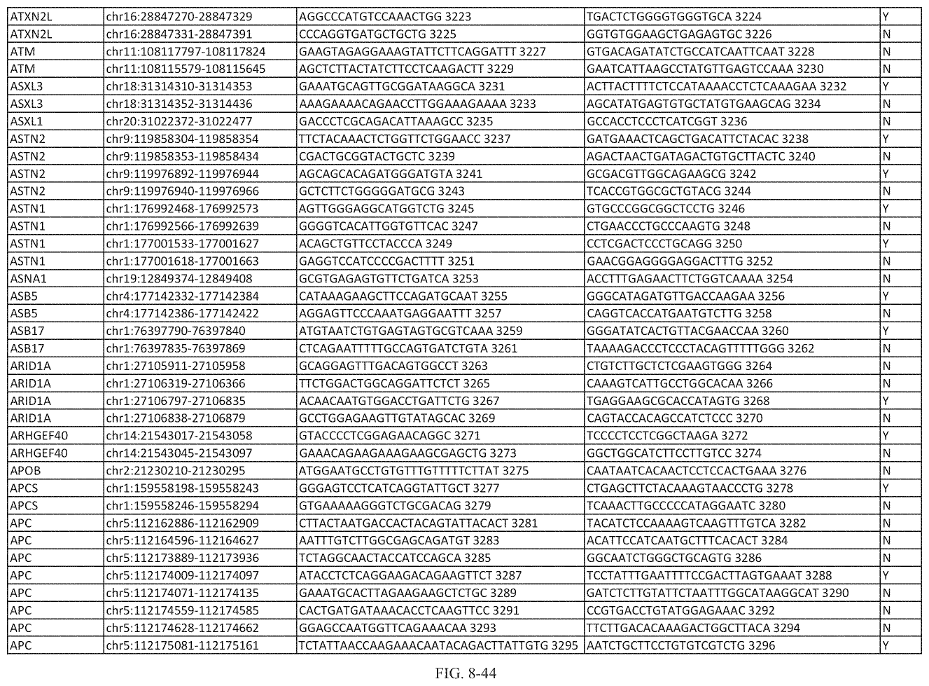

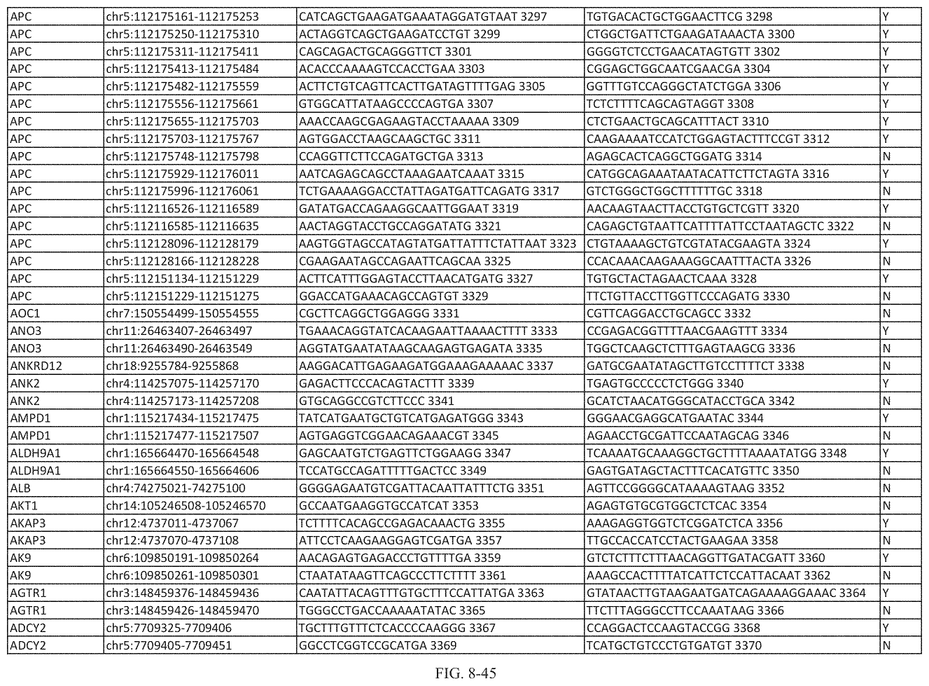

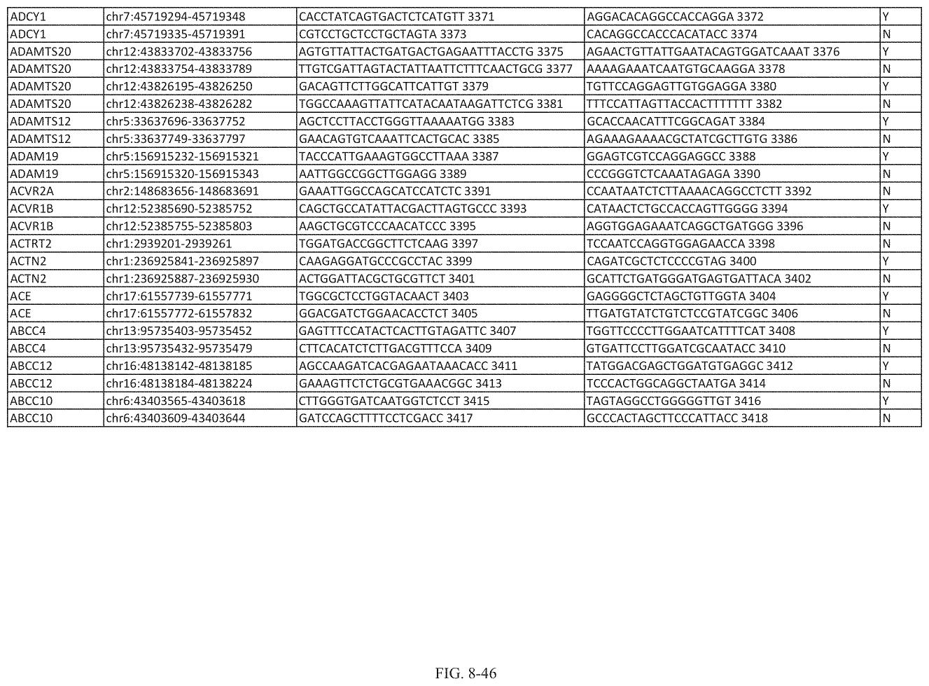

As a demonstrable example for off-the-shelf bank panels of the present invention, FIG. 8 shows a 1,688-amplicon panel targeting 7,118 unique variants. These variants were selected to design a lung cancer panel and they are estimated to target˜80% of the patients based on publicly available cancer genomics databases, such as The Cancer Genome Atlas (TCGA).

The target specific-primer pairs for each locus of interest (FIG. 8) were designed and identified using the procedures described in this application and VERSATILE® software (Pillar Biosciences, Inc.).

FIG. 8 shows a 1,688-primer panel targeting 7,118 unique variants of a lung cancer panel. All forward and reverse primers shown in FIG. 8 are target gene-specific primers without tags.

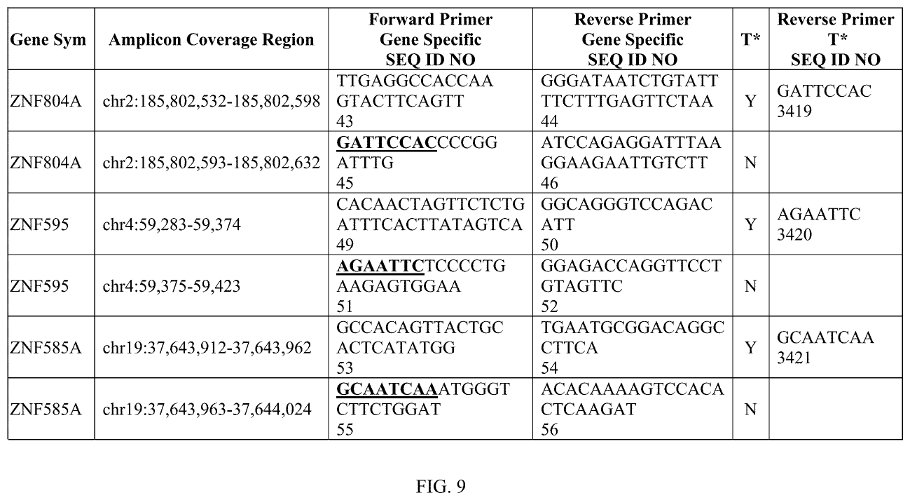

In the primer panel of FIG. 8, some amplicon regions have overlapping regions. Preferably, SLIMAMP® tags are added to inhibit the amplification of the overlapping target segments. FIG. 9 illustrates SLIMAMP® tags of 3 pairs of overlapping target genomic loci in FIG. 8. SLIMAMP® tag is added at the 5′-end of the first reverse primer of each pair. SLIMAMP® tag is a partial sequence of the 5′-end sequence of the second forward primer of each pair.

### Example 9. Protocol for Selecting Patient-Specific Primer Pairs Using WES Data

WES data of tumor and matched normal from 2 individuals A and B were processed and sequenced in-house. The DNA was extracted from blood (normal) and formalin-fixed paraffin-embedded (FFPE) tumor tissues obtained from the two patients. WES data was generated by using Roche's HyperCap workflow for exome captured followed by sequencing on Illumina's NextSeq machine. The sequencing raw data were demultiplexed and converted to FASTQ by BCL2FASTQ. The subsequent FASTQ files were subjected to quality-based filtering, removing reads with average Phred-based quality of less than 15 and read length of less than 75 bp. The filtered FASTQ files were mapped to the human genome reference (hg19) using BWA and the alignment was post-processed using sambamba and summarized using custom written Python scripts. All variants were identified from the paired WES data using VarDict software (Pillar Biosciences, Inc.). Variants that are strongly or likely somatic in origin are then inferred by comparing the tumor variant calls with normal variant calls using the var2vcf_paired.pl script from the VarDict package. A series of optional parameters are applied to select the somatic variants based on VAF and sequencing quality. This resulted in 2-5 patient-specific somatic variants of interest (VOI) which are then used to identify 2-5 genomic loci.

For the 2 patient samples A and B, Table 8 shows the number of mutations identified in each patient and the mutations within that patient.

TABLE 8

| | Number of final | |
|---|---|---|
| Patient | somatic mutations | List of somatic mutations |
| Patient A | 2 | ATM 1010G > A.Arg337His |
| | | KRAS 35G > A.Gly12Asp |
| Patient B | 5 | OR4D5 373G > A.Ala125Thr |
| | | TP53 810T > A.Phe270Leu |
| | | ZNF536 3699G > C.Trp1233Cys |
| | | ZNF804A 2461C > A.Pro821Thr |
| | | COL19A1 358T > G.Trp120Gly |

We then used the amplicon bank of Example 8 with the filtered somatic mutations to select the following patient specific primer pairs as shown in Table 9.

TABLE 9

| | | Target-Specific | Target-Specific | |
|---|---|---|---|---|
| | | Forward Primer Sequence | Forward Primer Sequence | Somatic mutations |
| Pat | Gene ID | (SEQ ID NO) | (SEQ ID NO) | covered |
| A | ATM | GAAGTAGAGGAAAGTAT | GTGACAGATATCTGC | 1010G>A.Arg337His |
| | | TCTTCAGGATTT 3227 | CATCAATTCAAT 3228 | |
| A | KRAS | ATCATATTCGTCCACAAA | TTTATTATAAGGCCTG | 35G>A.Gly12Asp |
| | | ATGATTCTGAATT 1957 | CTGAAAATGACTGA 1958 | |
| B | OR4D5 | TGACTGTCATGGCGTATG | GTGCACAGACAGTCTG | 373G>A.Ala125Thr |
| | | ACC 1409 | ATTCAT 1410 | |
| B | TP53 | CCCTTTCTTGCGGAGATT | CTTACTGCCTCTTGCTTC | 810T>A.Phe270Leu |
| | | C 399 | TCTTT 400 | |
| B | ZNF536 | AGGGACTGGTCTCACCT | CGCTCCGGCTTTGGCAG 70 | 3699G>C.Trp1233Cys |
| | | TTA 69 | | |
| B | ZNF804A | TTGAGGCCACCAAGTACTT | GGGATAATCTGTATTTTC | 2461C>A.Pro821Thr |
| | | CAGTT 43 | TTTGAGTTCTAA 44 | |
| B | COL19A1 | TAGCTGCCATGTTTCGAGT | CACAACTCTAATGGTACT | 358T>G.Trp120Gly |
| | | ACG 2819 | TTACCTGTG 2820 | |

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point to out and distinctly claim the subject matter regarded as invention, the following claims conclude the specification.

What is claimed is:

1. A method for detecting circulating tumor nucleic acid (ctTNA) from a liquid biological sample of a patient, comprising the steps of:
a. preparing cell free total nucleic acid (cfTNA) from the sample obtained at one or more timepoints from a patient who had cancer at an initial timepoint,
b. selecting at least 2-45 individualized primer pairs from a primer bank,
wherein the selected individualized primer pairs correspond to at least 2-45 genomic loci of one or more cancer genes, each primer pair is designed to amplify its corresponding genomic locus; wherein said at least individualized 2-45 genomic loci are selected in silico by (i) analyzing sequence data of whole genome, or whole exome, or a large gene panel, or specific cancer genes, of a tumor tissue of the patient, at the initial timepoint by a computational approach, and (ii) choosing at least 2-45 genomic loci containing somatic mutations specific for the patient, based on clonality, detectability, and frequency of the mutation;
wherein the primer bank is designed to comprise multiple primer pairs to amplify different genomic loci of a human, and the primer pairs in the primer banks are designed to be compatible in a single oligonucleotide pool by minimizing primer dimer interaction and reducing amplification of overlapping regions of amplicons by stem-loop inhibition during a PCR reaction;
c. adding the at least 2-45 individualized primer pairs into the cfTNA and perform gene-specific multiplex PCR reaction to amplify the selected genomic loci,
d. purifying, indexing, quantifying, normalizing, and sequencing the amplified DNA, and
e. analyzing the sequencing data and determining circulating tumor nucleic acid (ctTNA)—positive if variants are detected in at least one of the genomic loci in cfTNA.

2. The method of claim 1, wherein the at least 2-45 individualized primer pairs are 2-60, or 2-100, or 2-200, or 2-300 primer pairs.

3. The method of claim 1, the at least 2-45 individualized primer pairs are 2-45 primer pairs.

4. The method of claim 2, where in step (e), determining ctTNA positive if at least two of the genomic loci are detected in cfTNA.

5. The method of claim 1, wherein the primer bank is a physical bank comprising 50-50,000 primer pairs.

6. The method of claim 5, wherein the primer pairs in the physical bank are pre-made and pre-validated.

7. The method of claim 1, where step b(i) further comprises comparing the analyzed sequence date with matched germline DNA of the patient.

8. The method of claim 1, wherein the first primer of the primer pair comprises from 5′ to 3′ a first segment containing a first tag being a priming site for a subsequent amplification, and a second segment containing a first target-specific sequence complementary to one strand of a target DNA to be amplified.

9. The method of claim 7, wherein the first primer further comprises a UID between the first segment and the second segment.

10. The method of claim 7, wherein the second primer of the primer pair comprises from 5′ to 3′ a first segment containing a second tag being a priming site for a subsequent amplification, and a second segment containing a second target-specific sequence complementary to another strand of the target DNA to be amplified.

11. The method of claim 1, wherein the amplification of an overlapping region of the amplicons is inhibited by having the same tag at the 5′-end of the forward primer and the 5′—end of the reverse primer, wherein each of the forward primer and the reverse primer is complementary to the DNA of the overlapping region, and a partial sequence of the 5′-end portion of the forward primer is inserted in between the reverse primer and the tag.

12. The method of claim 1, wherein the liquid biological sample is blood, saliva, urine, sweat, or cerebrospinal fluid.

13. The method of claim 1, wherein the cfTNA is cfDNA, cfRNA, or the combination thereof.

14. The method of claim 13, wherein the cfTNA is cfDNA.

15. The method of claim 1, further comprising a step (f): determining a therapy choice or a change in therapy for the patient.
